(19) United States
(12) Patent Application Publication
(10) Pub. No.: US 2005/0032129 A1
(43) Pub. Date: Feb. 10, 2005

(54) Detection of antigen by immunohistochemical staining

(75) Inventor: Kazuhisa Hasui, Kagoshima City (JP)

Correspondence Address: OLIFF & BERRIDGE, PLC, P.O. BOX 19928, ALEXANDRIA, VA, 22320, US

(73) Assignee: KAGOSHIMA UNIVERSITY, Kagoshima City (JP)

(21) Appl. No.: 10/872,462

(22) Filed: Jun. 22, 2004

(30) Foreign Application Priority Data
Jun. 26, 2003 (JP) 2003-183,193

(51) Int. Cl. G01N 33/53; G01N 33/567; G01N 33/554; G01N 33/569

(52) U.S. Cl. 435/7.2; 435/7.32

(57) ABSTRACT

The object of the present invention is to provide a method for detecting antigens whereby it is possible to greatly reduce the time required for the detection of antigens, and improve the selection of proper dyes for the detection of given antigens, and which is suitable for the automated detection of antigens. A method for detecting two or more antigens based on multiplicated immunohistochemical staining using colored products or fluorescent dyes for visualizing the multiple antigens is characterized that an inhibitory step against non-specific reactions is performed for coating, with a non-specific reaction inhibiting substance, non-specific reactive substances which would otherwise react with primary antibodies in a non-specific manner, thereby inhibiting the non-specific reaction of those non-specific reactive substances with primary antibodies.

### BACKGROUND OF THE INVENTION

1. Field of the Invention

The present invention relates to a method for detecting antigens, particularly to a method for detecting antigens by multiplicated immunohistochemical staining.

2. Description of the Related Art

With the recent development of immunological science, immunoassay capable of detecting trace substances at a high sensitivity based on antigen-antibody reactions has been widely used. The generally used immunoassay includes immuno-histochemistry and enzyme immunoassay.

Immuno-histochemistry is a method for detecting a target antigen on a tissue by means of an antibody capable of specifically recognizing the antigen. Usually, a sampled tissue, after fixation, is embedded in paraffin, the paraffin-embedded tissue block is cut into slices, an antibody capable of recognizing a specific antigen is applied to the slices to bind the antibody to the antigen by means of an antigen-antibody reaction, and thus it is known whether a given tissue slice contains the target antigen by detecting the presence of antibodies bound to the tissue as a result of antigen-antibody reaction. The antibody which is allowed to first react with the target antigen is called a primary antibody. When a marker substance capable of emitting a signal detectable by sight or with equipment is linked to a primary antibody, it is possible to know the amount of bound antibodies by determining the intensity of the signal emitted by the bound antibodies which corresponds to the amount of reacted antigens present on a studied tissue. The marker substance capable of emitting a signal suitable for this purpose includes fluorescent dyes, enzymes, etc.

Since it is currently possible to analyze colored specimens with an optical microscope, enzymes that deposit a pigment have been generally used as a marker substance. The immuno-histochemistry consists of linking an enzyme to a primary antibody, applying, to a reaction system, a substrate that develops a color after reacting with the enzyme linked to bound antibodies, and assaying the intensity of a colored reaction product. The assay result represents the amount of bound antibodies, which corresponds to the amount of the target antigens present on the studied tissue. However, generally, this method does not have a sufficient sensitivity.

The immunohistochemical staining for the detection of antigen includes direct methods in which a primary antibody is labeled with an enzyme or marker which can be used for visualizing bound antibodies, and indirect methods in which a secondary antibody that reacts a primary antibody is labeled with a marker (Watanabe et al., “Enzyme immunoassay,” Gakusai Planning, Feb. 18, 2002, Revised 4th edition, pp. 23-25 (ref. 1)). Generally, the indirect immunohistochemical staining includes the followings. The peroxidase-antiperoxidase method (PAP method hereinafter) consist of labeling a secondary antibody with a maker, such as peroxidase and biotin, and labeling the primary antibody bound to the target antigens by means of the maker-labeled secondary antibody, amplifying the signal of the secondary antibody by means of anti-maker antibody labeled with peroxidase in the PAP method or streptavidin-peroxidase complex in sABC method, and visualizing peroxidase in hydrogen peroxide-pigment disposition reaction. The polymer reagent method is based on a reaction involving a polymer comprising a secondary antibody and a marker linked thereto. In addition, there are methods using an FITC (fluorescein isothiocyanate) or HRP (horseradish peroxidase) labeled primary or secondary antibody. There is also a ultra-high sensitive immunohistochemical staining based on the sABC method combined with catalyzed reporter deposition (CARD hereinafter).

The PAP method is outlined in ref. 1. An illustrative example of this method consists of binding a rabbit primary antibody to a tissue slice, and applying an excess amount of a pig anti-rabbit antibody to the tissue slice so that the pig anti-rabbit antibodies bind to the rabbit antibodies (ref 1, pp. 136-138). The pig anti-rabbit antibody has, at one of the two binding sites towards the rabbit antibody, HRP linked via antigen-antibody reaction with a rabbit anti-HRP antibody existing as a soluble complex (PAP complex). Such an HRP-labeled pig anti-rabbit antibody is applied to the tissue slice. Then, an aqueous solution of hydrogen peroxide, and a coupling pigment such as diaminobenzidine are applied to the reaction system to allow it to undergo a coloring reaction. A hematoxylin solution is applied to the tissue slice to stain cell nuclei which form a contrastive background. The slice is passed through a series of increasingly concentrated aqueous solutions of ethanol, to be dehydrated. The slice is immersed in xylene, covered with a glass plate, and sealed with a plastic solvent.

The sABC method is described in ref. 1 (pp. 138-144). According to this method, a fixed, paraffin-embedded tissue slice is removed of paraffin, immersed in a mixture comprising 0.03% aqueous solution of hydrogen peroxide and methanol for 20 minutes (ref. 1, p. 163) to block the activity of endogenous peroxidases, and transferred in a phosphate buffer to be made hydrophilic. Primary antibodies are allowed to bind to antigens present on the sliced tissue. Then, biotin-linked secondary antibodies are allowed to bind to bound primary antibodies. The system is then labeled with a streptoavidin-HRP complex. A mixture comprising an aqueous solution of hydrogen peroxide and a coupling pigment such as diaminobenzidine is applied to the tissue slice to cause a coloring reaction to occur at antigen-located sites. Hematoxylin solution is applied to the tissue slice to stain cell nuclei which serve as a contrastive background. The slice is passed through a series of increasingly concentrated aqueous solutions of ethanol, to be dehydrated. The slice is immersed in xylene, covered with a glass plate, and sealed with a plastic solvent.

The immuno-histochemistry using an HRP-labeled, anti-FITC antibody is outlined in ref. 1(p. 158). According to this method, a fixed tissue slice is made hydrophilic as in the sABC method, and then FITC-labeled primary antibodies are allowed to bind to antigens present on the tissue. Alternatively, FITC-labeled secondary antigens are allowed to bind to primary antibodies bound to antigens present on the tissue. Then, enzyme-labeled, e.g., HRP-labeled anti-FITC antibodies are allowed to bind to the FITC-labeled antibodies, and an appropriate substrate is applied to the system to cause HRP-based coloring reaction to occur so that the antigens present on the tissue can be detected indirectly. Zymed advertises, in their Web site, a kit for this application (see an advertisement of an NBA kit (non-biotin assay kit) in the Web site of Zymed at http://www.zymed.com/, or http://www.zymed.com/pindex/index9.html (ref. 2)).

A polymer-reagent method is disclosed in Japanese Patent Application Publication No. 2001-181299 (ref. 3)). A polymer serves as a carrier to support an enzyme (e.g., HRP) and an antibody (protein) thereon, and ref. 3 states that this complex enables the highly sensitive detection of a substance specifically bound to the antibody. Claim 13 of ref. 3 outlines the polymer reagent method. The procedures of the polymer reagent method as described below are outlined in ref. 1 (p. 147). A fixed tissue slice is made hydrophilic as in the sABC method. An antibody is allowed to specifically bind to an antigen present on a tissue slice. Then, a polymer complex carrying a secondary antibody and an enzyme such as HRP is allowed to bind to the primary antibody specifically bound to the antigen, and an HRP-based coloring reaction is allowed to occur so that the antigen can be indirectly detected. For this purpose, DakoCytomation provides Envision and ChemMate Envision, while Nichirei provides Simple Stain System. DakoCytomation also provides Epos System which uses a polymer complex comprising a specific antibody and an enzyme, instead of a secondary antibody.

The CARD method is described in U.S. Pat. No. 5,731,158 (ref. 4). Claim 9 of ref. 4 outlines the use of biotin tyramine or fluorescein (e.g., FITC)-linked tyramine. Claim 9 also outlines CARD using biotin tyramine. According to the description of ref. 1 (pp. 150-152) and Bobrow, M. N. et al., “Catalyzed reporter deposition, a novel method of signal amplification. Application of immunoassays,” J. Immunol. Methods, Dec. 20, 1989, 125(1-2): 279-85 (ref. 5), it is possible to achieve the ultra-high sensitive detection of a target substance by utilizing the deposition of a marker substance specifically bound to the target substance occurring as a result of enzymatic reaction which ensures the amplification of detection sensitivity. Adams introduced 1992 CARD reaction using biotin tytamide to histochemistry (Adams, J C, “Biotin amplification of biotin and horseradish peroxidase signals in histochemical stains.” J Histochem Cytochem. 1992 October;40(10):1457-63). Merz, H. et al. report (Merz, H. et al., “ImmunoMax. A maximized immunohistochemical method for the retrieval and enhancement of hidden antigens,” Lab. Invest., July 1995, 73(1):149-56, (ref. 6) that, when antigen-retrieval treatment (ref. 1) is applied, in combination with marker signal amplification (ImmunoMax), to a chemically fixed, paraffin-embedded tissue slice, it is possible to detect antigens that are unresponsive to a method solely dependent on simple antigen-antibody reaction. Refs. 5 and 6 further introduce the procedures of a certain ultra-high sensitive immunohistochemical method. According to their descriptions, a fixed tissue slice is made hydrophilic as in the sABC method, antigens are retrieved, the antigens are labeled by the sABC method, HRP is used to deposit biotin tyramine, and detection sensitivity of the labeled antigens is amplified using a streptoavidin-HRP complex. This ultra-sensitive immunohistochemical method can detect a target antigen at a sensitivity 1000 times higher than that of the sABC method. This method is named as ImmunoMax, and for the practice of this method, DakoCytomation provides a Catalyzed Signal Amplification (CSA) system. With this method, however, non-specific reaction due to endogenous biotin is considerable associated with the retrieval of antigens. Ref. 1 (pp. 164-165) describes a newly developed method for masking the endogenous biotin by immersing a tissue sample in a 0.1% avidin solution and then in a 0.01% biotin solution. A modified ImmunoMax method is developed as described in Hasui K., et al., “Quantitative highly-sensitive immunohistochemisty (modified ImmunoMax) of HTLV-1 p40 tax and p27rex proteins in HTLV-1-associated non-neoplastic lymphadenopathy (HANNLA) with estimation of HTLV-1 dose by polymerase chain reaction,” Dendritic Cells, 1997, 7:19-27, (ref. 7), and Hasui, K., “A study on the ultra-high sensitive immunohistochemistry for the detection of HTLV-1 in HTLV-1 associated diseases,” Summary of the research (Basic Research C-2, No. 10670166) financially supported by the Ministry of Education, Japanese Government from fiscal 1998 to 1999 (ref. 8). HRP is used to deposit biotin tyramine, and detection sensitivity of the labeled antigens is amplified using a streptoavidin-HRP complex. This ultra-sensitive immunohistochemical method can detect a target antigen at a sensitivity 1000 times higher than that of the sABC method. Zymed provides a kit for this ultra-high sensitive immunohistochemical method using no biotin. The U.S. Pat. No. 6,203,989 (ref. 9) discloses, for visualizing a signal from a target nucleic acid labeled by in-situ hybridization, the use of a marker such as a fluorescent dye (e.g., FITC), in addition to the use of an enzymatic reaction based on HRP, alkaliphosphatase, etc. Claim 9 of ref. 4, and ref. 9 outline the use of a fluorescent dye for labeling tyramine deposits in CARD.

Nowadays, conventional multiplicated immunohistochemical methods for detecting two or more antigens on a fixed, paraffin-embedded tissue slice consist, when the detection of two antigens (first and second antigens hereinafter) is considered for illustration, of performing a first staining which includes the deposition of a colored reaction product (coupling pigment hereinafter) or a fluorescent reaction product effective for the detection of the first antigen, and then washing away immuno-reacted substances other than the deposited coupling pigment or fluorescent product, by immersing the tissue slice in a buffer with pH adjusted to pH 3 or lower, or pH 9 or higher, and of subjecting the sliced tissue to a second staining for the detection of the second antigen.

For the outline of the conventional multiplicated immuno-hisotochemistry and its procedures, see the Web site of Zymed (ref. 2). According to the information obtained from this Web site, suitable buffers of pH 3 or lower or pH 9 or higher used for the removal of immuno-reactive substances other than a deposited coupling pigment or fluorescent dye generally include hydrochloride-buffered 0.1M glycine solution with pH 2.2. A tissue slice, having undergone a first staining for labeling a first antigen, is immersed in this buffer, and is kept there for several hours at room temperature with stirring while the solution is replaced at least three times. Ref. 1 describes that the antigen retrieval may be achieved by heating. Specifically, ref. 1 (p. 195) describes that the antigen retrieval by heating enables the inactivation or removal of immuno-reactive background substances other than an immuno-reactive product effective for the detection of a first antigen which is linked with a coupling pigment or fluorescent dye. Generally, this treatment consists of immersing a fixed, paraffin-embedded tissue slice having undergone a first staining for the detection of a first antigen, in 0.01M citric acid buffer with pH 6.0, and heating the tissue slice by transferring it to a stirrer equipped with a heater or to an autoclave. DakoCytomation provides a kit for practicing the heating-based antigen retrieval so as to inactivate or remove immuno-reactive background substances other than an immuno-reactive product effective for the detection of a first antigen which is linked with a coupling pigment or fluorescent dye.

Alternative methods for antigen retrieval may include followings. One method is to utilize the saturation condition of an antigen-antibody reaction utilized in the immunohistochemical staining. A second method consists of heating a tissue slice having undergone a first staining, thereby inactivating or removing immuno-reactive background substances other than an immuno-reactive product effective for the detection of a first antigen which is linked with a coupling pigment or fluorescent dye, and applying, to the tissue slice, a second staining which uses, for the detection of a second antigen, alkaline phosphatase instead of HRP as an enzyme necessary for the coloration of another coupling pigment or fluorescent dye.

The conventional multiplicated immunohistochemical staining includes further variations. Such a variant method uses, for the detection of first and second antigens, two different antibodies (primary antibodies hereinafter) specifically directed to the two antigens. A second method uses two different reactions, such as antigen-antibody reactions or chemical reactions, specifically directed to the two antigens which are labeled by the same marker, e.g., FITC, stains first and second antigens simultaneously, and discriminates between the two antigens dependent on their specificity to the respective reactions.

The aforementioned immuno-histochemistry used for the detection of two or more antigens is called multiplicated immuno-histochemistry. The multi-plicated immuno-histochemistry for the detection of three or more antigens, however, has little practical meaning. The multiplicated immuno-histochemistry for the detection of two antigens which is practically meaningful is called duplicate immuno-histochemistry. Generally, the duplicate immuno-histochemistry is used for characterizing the two antigens which have been identified in preceding immunohistochemical studies.

In immuno-histochemistry, non-specific reactions should be avoided as far as possible. This is because non-specific immuno-reactive substances other than a target antigen are stained as well which, unless inactivated or removed, might be interpreted finally as part of the target antigen.

Such non-specific reactions may be brought out by factors endogenous to a fixed tissue slice, processes the tissue slice receives, reagents used in staining processes, washings subsequent to staining processes, staining processes themselves, etc.

As a measure to inhibit such non-specific reactions, a known method is directed to inhibit endogenous peroxidase (ref. 1, p. 163). Non-specific re-actions due to endogenous peroxidase are one of the non-specific reactions due to factors endogenous to a fixed tissue slice. Inhibition of endogenous peroxidase is achieved by immersing a fixed, paraffin-embedded tissue slice in a 0.03% solution of hydrogenperoxide in ethanol, before the tissue is removed of paraffin and made hydrophilic for 20 minutes, or in a phosphate buffer containing hydro-genperoxide at 0.3% for five minutes after the tissue slice is made hydrophilic.

Non-specific reactions of antibodies may be classified to non-specific reactions resulting from the reagents used in various reactions, and non-specific reactions occurring as a result of washings subsequent to reactions. In order to inhibit non-specific reactions which may occur during staining, a known method consists of employing fractionated immunoglobulins as specific (primary) and secondary antibodies. According to the disclosure of the U.S. Pat. No. 5,869,274 (ref. 10), it is possible to inhibit non-specific reactions of antibodies by applying monovalent antibodies to a studied tissue before the tissue undergoes specific reaction. Ref. 10 and ref. 1 (p. 43) describe that non-specific reactions can be inhibited by adding a serum sample extracted from an animal such as horse, sheep or rabbit which contains an antibody corresponding to a primary antibody to a diluted solution of a specific antibody. Ref. 1 (p. 115) further adds that it is possible to inhibit non-specific reactions by applying a 0.25% casein solution to a tissue slice for 5-30 minutes before the tissue slice undergoes antigen-antibody reaction. Tris-buffered solutions supplemented with a detergent such as 0.1% Tween 20 are marketed as an antibody diluent.

Non-specific reactions requiring the introduction of antigen retrieval result from the processes applied to a fixed tissue slice, and pose a problem in ultra-high sensitivity immuno-histochemistry. They are largely accounted for by endogenous biotin which requires the introduction of antigen retrieval. Ref. 1 (pp. 164-165) mentions a known method for inhibiting non-specific reactions which consists of treating a tissue slice with a 0.1% avidin solution, thereby masking endogenous biotin, and then treating the slice with a 0.01% biotin solution, thereby further masking avidin remaining in the tissue. This method is applied to a ultra-high sensitive immuno-histochemistry incorporating the sABC method. See ref. 7. Ref. 8 points out non-specific reactions in the ImmunoMax method (as described in ref. 2), and asserts that it is possible to realize ultra-high sensitivity immuno-histochemistry by performing biotin masking using a conventional method based on the combination of avidin and biotin solutions, and modifying washing conditions subsequent to reactions. Ref. 8 (pp. 3-9) describes that the detection sensitivity of labeled products is amplified 1000 folds by means of the HRP-based labeling of biotin tyramine deposits, and further informs of the optimum condition for the introduction of endogenous biotin masking in ultra-high sensitive immuno-hitochemistry, washings subsequent to staining processes, and conditions for the successful inhibition of non-specific reactions subsequent to fixation.

In immuno-histochemistry, washing is carried out with a phosphate buffer, tris buffer, or tris buffer supplemented with a surfactant. In immuno-histochemistry, each time a reaction is completed, washing is carried out by immersing a tissue sample in a buffer for five minutes, and repeating this session three times with a renewed buffer. Refs. 7 and 8 note that, in ultra-high sensitive immuno-histochemistry incorporating the sABC method, it is necessary to perform thorough washing during the practice of the sABC method, and that, since the deposition of biotin tyramine in CARD is non-specific, washing is performed for removing residual non-deposited biotin tyramine, and washing must be also introduced subsequent to the labeling of deposited biotin tyramine with HRP-linked streptoavidin. Refs. 7 and 8 further note that, when a staining system based on a capillary-gap method is used, washing to be performed during the practice of the sABC method should be done by using a tris buffer supplemented with a surfactant and heated to 35° C., and that washing with a tris buffer kept at room temperature is necessary subsequent to the HRP-base labeling of biotin tyramin deposits.

For the standardized practice of immuno-histochemistry, automated immunohistochemical staining systems have been developed and widely used. The capillary gap method consists of placing two glass slides each carrying a fixed tissue slice thereon opposite to each other, and allowing the gap between the two slide glasses to absorb, via capillary action, reaction or washing solutions, and removing the reaction or washing solutions by means of an absorbing material after reaction or washing. These operations are performed under the control of a computer. According to a drop-type computer-controlled system, micro-pump driven syringes aspirate reaction or washing solutions stored in specified places, and apply them to a fixed tissue slice mounted on a slide glass placed horizontally. The automated drop-type staining system resembles a manual operator working on a bench, and has been widely used. Another automated system comprising a base plate allowing temperature-adjusted heating has been developed. With this system, a slide glass carrying a tissue slice on its surface is placed with respect to the base plate so as to allow the tissue slice to directly face the base plate with a thin gap in between, and reaction or washing solutions are allowed to flow through the gap, so that reactions or washings can be performed under temperature control.

The automated immunohistochemical staining system is a system for staining a tissue slice immunohistochemically under the control of a computer. The automated immunohistochemical staining system can be classified according to the manner how a slide glass carrying a tissue slice is set to the system, into two types, i.e., capillary-gap type and drop-type. With the capillary-gap type system, two slide glasses placed opposite with a thin gap in between, and reaction reagents are absorbed into the gap via capillary action. With the drop-type system, reaction reagents are applied dropwise on a tissue slice placed on a slide glass. Some systems comprise a computer in which a program is installed for adjusting the duration of reactions, and the manner of washings subsequent to reactions. With other systems, distribution of reaction reagents to tissue slices, duration of reactions, and manner of washings subsequent to reactions can be adjusted as appropriate. The former is for applying a specified immunohistochemical staining to tissue slices (fixed type system) and generally is provided together with reagents and washing agents. The latter is for applying an immunohistochemical staining designed by the user to tissue slices (on-demand type system). The U.S. Pat. No. 6,349,264 (ref. 11) describes an automated immunohistochemical staining system in which reagents are applied dropwise to horizontally arranged slides under the control of a computer. Ref. 11 also mentions an on-demand type system. Hashizume et al. (Hashizume, K. et al., “Automated immunohistochemical staining of formalin-fixed and paraffin-embedded tissues using a catalyzed signal amplification method,” Appl. Immunohistochem. Mol. Morphol., 9(1):54-60, March 2001 (ref. 12)) mentions it is possible to provide an automated immunohistochemical staining system with a ultra-high sensitivity (based on catalyze signal amplification (CSA) system) which performs heating-based antigen retrieval using a heater-equipped stirrer unit, and masks background biotin using avidin and biotin solutions. Ref. 12 describes that the automated immunohistochemical staining system enables ultra-high sensitive immunohistochemical staining. In the Web site http://www.ventanadiscovery.com/product/index.html (ref. 13), there is a description about an automated immunohistochemical staining system (The Ventana Discovery) with a thermo-controlled base plate in which a slide glass carrying a tissue slice on its surface is placed with its tissue slice bearing surface facing the base plate with a thin gap in between, and reaction or washing solutions are allowed to pass through the gap, thereby allowing reactions or washings to be performed on the tissue slice under temperature control.

With the aforementioned conventional classical multiplicated immunohistochemical methods, removal of immuno-reactive background substances other than a coupling pigment or a fluorescent dye deposited on a sample using a hydrochloric acid buffered 0.1M glycine with pH 2.2 requires several hours at room temperature.

Moreover, there is no reliable signal or criteria for indicating the complete removal of non-specific immuno-reactive substances. HRP has been used in combination with plural substrates for activating different coloring pigments, but which combination gives rise to which color pigment has not been clearly defined. The kit provided by DakoCytomation requires heating during the staining process and thus its installment in an automated staining system may be difficult.

### SUMMARY OF THE INVENTION

The object of the present invention is to provide a method for detecting antigens whereby it is possible to greatly reduce the time required for the detection of antigens, and improve the selection of proper dyes for the detection of given antigens, and which is suitable for the automated detection of antigens.

The present inventors tried hard to inhibit the non-specific reactions of primary antibodies, and studied the effects of various buffer solutions or inhibitory substances on such non-specific reactions, and succeeded in finding the inventive method suitable for detecting antigens. The inventive method is a method for detecting two or more antigens based on multiplicated immunohistochemical staining which uses colored products or fluorescent dyes for visualizing multiple antigens. According to the inventive method, an inhibitory step against non-specific reactions is introduced to coat disturbing substances which would otherwise react with primary antibodies in a non-specific manner to give rise to non-specific reaction products, thereby inhibiting the non-specific reaction of those disturbing substances with primary antibodies.

According to a preferred embodiment of the inventive method for detecting antigens, the inhibitory step against non-specific reactions is performed before a primary antibody is allowed to react with a first antigen.

According to another preferred embodiment of the inventive method for detecting antigens, the inhibitory step against non-specific reactions comprises treating a test sample with at least one chosen from the group comprising serum derived from an animal species from which a secondary antibody is prepared, skimmed milk, non-fat milk and casein solution.

The casein treatment uses a solution containing casein at 0.025 to 2.5%.

According to yet another preferred embodiment of the inventive method for detecting antigens, immuno-reactive substances bound to antibodies other than immuno-reactive products linked to colored products or fluorescent dyes used for the detection of antigens other than the lastly detected antigen, are removed by means of a buffer with a pH adjusted to pH 3 or lower, or pH 9 or higher.

According to yet another preferred embodiment of the inventive method for detecting antigens, the method further comprises a step for cleaning a test sample using heated cleaning solution so that the test sample is removed of non-specific reaction products and other residual reaction products.

According to a preferred embodiment of the inventive method for detecting antigens, the temperature of the heated cleaning solution is in the range of 25 to 60° C.

According to another preferred embodiment of the inventive method for detecting antigens, removal of immuno-reactive substances other than immuno-reactive products linked to colored products or fluorescent dyes, is performed each time after the detection of an antigen till the detection of the last antigen.

According to yet another preferred embodiment of the inventive method for detecting antigens, the immunohistochemical staining uses plural pigments having different component profiles, and plural antigens are discriminated based on the different component profiles of the pigments.

According to yet another preferred embodiment of the inventive method for detecting antigens, the different component profile of plural pigments is expressed as a combination of R (red), B (blue) and G (green) components.

According to yet another preferred embodiment of the inventive method for detecting antigens, the colored product or fluorescent dye is a substance allowing visualization.

According to yet another preferred embodiment of the inventive method for detecting antigens, the substance allowing visualization comprises a coupling substance which becomes colored or emits light as a result of an immunohistochemical reaction where a labeling enzyme reacts with a substrate.

According to yet another preferred embodiment of the inventive method for detecting antigens, the substance allowing visualization is chosen from the group comprising horseradish peroxidase (HRP), alkaliphosphatase (ALP), glucose oxidase, and beta-galactosidase.

According to yet another preferred embodiment of the inventive method for detecting antigens, a program is designed for the execution of test procedures such as the selection of reaction solutions, reaction durations and washing times, and installed in an automated immunohistochemical staining system for the automated execution of immunohistochemical staining.

### BRIEF DESCRIPTION OF THE DRAWINGS

FIG. 1 shows diagrams for illustrating final reaction products which are distinguished by optical microscopy according to an embodiment of the inventive immunohistochemical staining including duplicate staining.

FIG. 2 shows the photographic images of tissue slices of a human tonsilla stained with DAB. These images are presented for illustrating the effect of the acidified glycine in the removal of non-specific primary antibody products, that is, the effect of removal of non-specific primary antibody products on the detection of a test antigen.

FIG. 3 shows the photographic images of tissue slices of a human tonsilla stained with DAB. These images are presented for illustrating the masking effect of the acidified glycine against the non-specific binding of primary antibodies to antigens.

FIG. 4 shows the photographic images of tissue slices of a human tonsilla. These images are presented for illustrating the actual coloration of coupling agents elicited by HRP activation.

FIG. 5 shows the photographic images of tissue slices of a human tonsilla. These images are presented for illustrating the duplicate immunohistochemical staining based on the combination of two coupling pigments activated by HRP which are respectively linked to antibodies specifically directed to CD68 and Fascin antigens (the two antigens are partially located at the same sites).

FIG. 6 shows the photographic images of tissue slices of a lymph node. These images are presented for illustrating the duplicate immunohistochemical staining based on the combination of two coupling pigments DAB and VIP both activated by HRP which are respectively linked to antibodies specifically directed to CD3 and CD5 antigens (the two antigens are partially located at different sites).

FIG. 7 shows the photographic images of tissue slices of a human tonsilla. These images are presented for illustrating the duplicate immunohistochemical staining based on the combination of two coupling pigments DAB and VIP both activated by HRP which are respectively linked to antibodies specifically directed to CD3 and CD79a antigens (the two antigens are located at different sites).

FIG. 8 shows the photographic images of tissue slices of oral mucosa. These images are presented for illustrating the duplicate immunohistochemical staining based on the combination of two coupling pigments SG and AEC both activated by HRP which are respectively linked to antibodies specifically directed to p53 protein and Ki-67 antigens (the two antigens are located at different sites).

### DESCRIPTION OF THE PREFERRED EMBODIMENTS

First, the inventive method for detecting antigens will be outlined below. The inventive method comprises subjecting a tissue sample to following treatments: an antigen retrieval treatment (this treatment is not always necessary, but execution of this treatment improves the specificity of antigens and detection sensitivity towards target antigens), washing, non-specific reaction inhibiting treatment (during the reaction of a primary antibody with a first antigen), binding of an enzyme-linked secondary antibody to a primary antibody bound to the first antigen (complex formation), identification of the first antigen, and washing. This completes a session for the first antigen. The same session is repeated until all the target antigens are detected.

Usually, two or more antigens are found on a fixed tissue slice, and thus the inventive method may be applied for detecting two or more antigens on such a fixed tissue slice. Preparation of a fixed (paraffin-embedded) tissue slice will be described below. The fixed (paraffin-embedded) tissue slice is prepared from a tissue sample extracted from a tissue of a living organism, to be fixed. Specifically, the sample is fixed, for example, chemically with 10% buffered formalin, passed through a series of increasingly concentrated ethanol solutions to be dehydrated, soaked with 100% xylene, then with paraffin melt kept at about 60° C., and embedded in solid paraffin which has been cooled to be solidified. The fixed (paraffin-embedded) tissue slice is obtained by cutting the above paraffin-embedded tissue block with a microtome into thin slices having a thickness of about 3 μm. The cut tissue slices are glued on slides appropriately treated. When it is required to stain a fixed, paraffin-embedded tissue slice immunohistochemically, the fixed, paraffing-embedded tissue slice is soaked with 100% xylene to be removed of paraffin, xylene permeating the tissue slice is replaced with 100% ethanol, and the tissue slice is then soaked with a phosphate buffer to be hydrophilic.

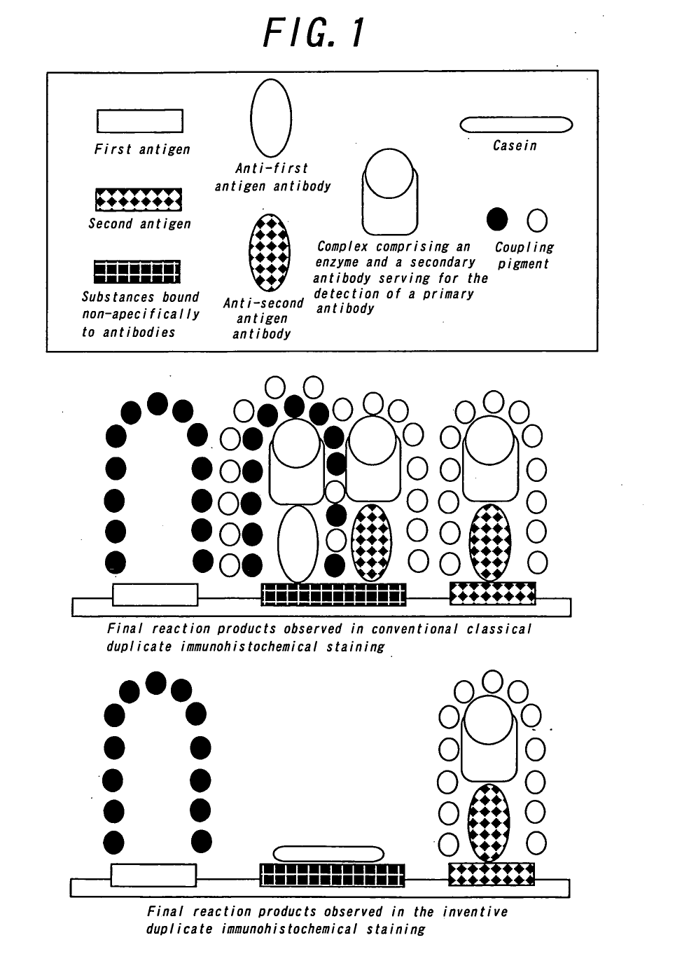

Next, the principle underlying the inventive method will be described. FIG. 1 is a diagram for showing reaction products observed during the detection of first and second antigens which are located at different sites of a tissue sample based on a conventional, classical duplicate immunohistochemical staining. FIG. 1 illustrates immunohistochemical staining for the detection of two antigens for the simplicity of illustration, but the inventive method is not limited to immunohistochemical staining for the detection of two antigens, but may include multiplicated immunohistochemical staining effective for the detection of two or more antigens. According to the conventional method, enzyme-linked primary antibodies are allowed to bind to first antigens, but a tiny fraction thereof are bound to non-specific reactive substances to form a complex, and those bound complexes will be removed thereafter. However, not all non-specific primary antibody complexes are removed by this treatment, and thus complexes comprising an enzyme-linked anti-second antigen primary antibody bind to such residual non-specific primary antibody complexes, which causes the non-specific primary antibody complexes to be stained with two coupling pigments to be used for the separate detection of first and second antigens. In contrast, according to the inventive method, non-specific reaction inhibiting treatment is performed to inhibit the non-specific binding of primary antibodies, and thus the opportunity for the complexes comprising an enzyme-linked anti-second antigen primary antibody to meet the non-specifically bound primary antibodies are eliminated. Therefore, according to the inventive method, a first antigen is surrounded only by one kind of coupling agent, while a second antigen is surrounded only by a different kind of coupling agent which will be activated by an enzyme attached to a complex comprising a primary antibody as well. Even with conventional duplicate immunohistochemical stainings other than the classical duplicate immunohistochemical staining, if non-specific reactions are not deliberately inhibited, non-specific reactive substances will be doubly stained by two coupling pigments which should be used for the independent detection of first and second antigens. Since, according to the inventive method, non-specific binding of antibodies to background reactive substances other than target antigens is prevented, it is possible to detect only target antigens at a high sensitivity. Thus, the final products obtained on completion of duplicate immunohistochemical staining are different between the conventional method and the inventive method.

The inventive method for detecting antigens uses multiplicated immunohistochemical staining for distinguishing different antigens by staining them with multiple colored products or fluorescent dyes for their separate detection. Multiplicated staining used herein means staining two or more different antigens immunohistochemically using multiple pigments.

According to the inventive method, the same multiplicated immunohistochemical staining is performed to inhibit non-specific reactions, the inhibition being achieved by masking non-specific reactive substances which would otherwise bind to primary antibodies, thereby eliminating the opportunity for those substances to bind to primary antibodies.

The non-specific reaction inhibiting treatment used herein includes any non-specific inhibiting treatment which is performed with an intention to inhibit the binding of primary antibodies to reactive substances other than target antigens in a non-specific manner, and is not limited to any specific treatment. The non-specific reaction inhibiting treatment includes, for example, treatment of a test sample with at least one chosen from the group comprising casein, serum derived from an animal species from which a secondary antibody is prepared, skimmed milk, and non-fat milk. Skimmed milk or non-fat milk can be used particularly for masking the activity of enzymes contained in serum.

The casein treatment preferably uses a solution containing casein at 0.025 to 2.5%, more preferably 0.1 to 1.0%, most preferably 0.25±0.1%.

In fact, the inventive method enables the detection of plural antigens. When the inventive method is used for the detection of 1st (antigen detected first) to n'th antigens (antigen detected last) (n=1, 2, 3 . . . ), it is preferred to remove immuno-reactive substances bound to antibodies (1st to (n−1)th antigens) other than immuno-reactive products linked to colored products or fluorescent dyes used for the detection of antigens other than the lastly detected antigen, by means of a buffer with a pH adjusted to pH 3 or lower, or pH 9 or higher. The pH of the buffer should be pH 3 or lower, or pH 9 or higher. This is because antigen-antibody reaction is inhibited in those pH ranges. Thus, in that buffer, primary antibodies bound to non-specific reactive substance are dissociated from those substances, and thus non-specific reactive substances other than coupling pigments (colored products, fluorescent dyes) can be removed.

The immuno-reactive substances bound to antibodies (1st to (n−1)th antigens) other than immuno-reactive products linked to colored products or fluorescent dyes used for the detection of antigens other than the lastly detected antigen include, in the ABC method, primary antibodies, biotin-labeled secondary antibodies, enzyme-labeled avidin, etc. In the polymer method, they include primary antibodies, enzymes, polymers comprising a secondary antibody.

A preferred embodiment of the inventive method for the detection of antigens further comprises a step for washing a tissue sample with a heated washing solution to eliminate non-specific reaction products and other residual reaction products. This step also enables the removal of non-specific reaction products and other residual reaction products. The temperature of the washing solution is preferably in the range of 25-60° C. When the tissue slice is based in such a washing solution, antigen-antibody reaction occurs on the tissue which facilitates the removal of non-specific reaction products and other residual reaction products.

Removal of immuno-reactive substances other than immuno-reactive products linked to colored products or fluorescent dyes, is performed each time after the detection of an antigen till the detection of the last antigen. This is for minimizing the adverse effect due to a preceding step on the succeeding step. However, if combination of two sets of primary and secondary antibodies directed to their respective two antigens is specially chosen, as in the duplicate staining of two antigens CD68 and Facin (Example 4), the washing step may be dispensed with.

Immunohistochemical staining is a method for detecting an antigen on a tissue sample by applying, to the tissue sample, an antibody capable of specifically recognizing the antigen. The immunohistochemical staining of the present invention uses, for the detection of plural antigens, plural pigments having different component profiles, and the plural antigens are discriminated based on the different component profiles of the pigments. In principle, use of plural pigments having different component profiles makes it possible to detect plural antigens. How two pigments having different component profiles can be used for the detection of plural antigens will be described on the premise that DAB and DAB-Ni are used as two different pigments. An image of an antigen stained with DAB has a large R component and an image of an antigen stained with DAB-Ni has also a large R component (see Table 2). However, when the R component is compared with other components, it is shown that the R component is larger than the G and B components. In fact, when a DAB-stained digital color image is decomposed into R, G and B components, the G and B components are comparatively large. In contrast, with a DAB-Ni-stained image, the R, G and B components are comparable in magnitude. Thus, the component profile is effective for distinguishing plural antigens based on their color images.

According to a preferred embodiment, a color image of a tissue is decomposed into R (red), B (blue) and G (green) components. In this specification, color images of tissues are decomposed in R, G and B components to be displayed as plates. This is partly for illustration purposes. The manner of decomposing a colored image is not limited to the above method. A colored reaction product or fluorescent dye preferably exhibits a visible color.

The colored product or fluorescent dye is preferably a coupling pigment capable of developing a color or emitting light as a result of reaction with a labeling substance. More preferably, such a visible substance is a coupling substance capable of developing a color or emitting light as a result of reaction where a labeling enzyme reacts with a substrate. Such a visible substance is more preferred because a tissue slice stained with plural such stains allows combinational use of an optical microscope, fluorescent microscope and confocal laser microscope towards the slice.

The substance capable of emitting a visible signal includes horseradish peroxidase (HRP), alkaline phoshphatase (ALP), glucose oxidase, beta galactosidase, etc. The visualization supporting substance widely used includes HRP and ALP.

The coupling pigment which develops a color in a reaction system where a labeling enzyme reacts with a substrate includes, in a reaction system where HRP acts on hydrogen peroxide, diaminobenzidine (DAB) or an agent widely used, aminoethylcarbazole (AEC), as well as VIP, SG, NovaRed, and TMB provided by Vector. In a reaction system where ALP acts on naphthol AS-BI sodium phosphate, fast red, fast violet, fast blue, new fuchsin, 5-bromo-4-chloro-3-indoxylphosphate/nitroblue tetrazorium, etc. may be used.

A preferred embodiment of the present invention comprises designing a program for the execution of test procedures such as the selection of reaction solutions, reaction durations and washing times, and installing the program in an automated immunohistochemical staining system for the automated execution of immunohistochemical staining.

According to the invention, it is possible to install the above program in a commercial automated immunohistochemical staining system and to execute automated immunohistochemical staining in which the selection of reaction solutions, reaction durations and washing times are automated.

### EXAMPLE

The present invention will be described by means of examples, but it should be understood that the present invention is not limited to those examples.

### Example 1

The duplicate immunohistochemical staining for detecting first and second antigens includes in the inventive multiplicated immunohistochemical staining comprises 18 steps as described below.

1) A fixed, paraffin-embedded tissue slice is immersed in xylene and in ethanol to be removed of paraffin. The immersion occurs for 3 to 20 minutes and is repeated three to five times.

2) The tissue slice is immersed in a 0.3-6% solution of hydrogen peroxide in methanol for 5 to 30 minutes, to perform a first-time inhibitory treatment of endogenous peroxidase.

3) The tissue slice is washed with 0.005-0.02M phosphate buffer solution plus 0.5-1% aqueous solution of sodium chloride (PBS), to be hydrophilic.

4) The tissue slice is subjected to an antigen retrieval treatment appropriate for target antigens (usually, the tissue slice is immersed in 0.001-0.2M citric acid-buffered solution, transferred to an autoclave to be heated at 110 to 140° C. for 1 to 15 minutes, and transferred, after cooling, to 0.01-0.1M phosphate buffer solution plus 0.85% aqueous solution of sodium chloride (PBS)).

5) The fixed tissue slice is mounted on an automated immunohistochemical staining system.

6) The tissue slice is washed one to four times with 0.01-0.1M tris buffer solution supplemented with 0.5-1% aqueous solution of sodium chloride containing 0.01-0.2% poly(oxyethylene)sorbitan monolaurate (tween 20)(TBST).

7) The tissue slice is immersed in 0.1-1% casein solution to allow reaction to proceed for two to 30 minutes. This is a pretreatment for inhibiting the occurrence of non-specific reaction due to primary antibodies specifically directed to a first antigen (anti-(1st antigen) primary antibody).

8) An anti-(1st antigen) primary antibody is applied to the tissue slice at an optimum concentration for an optimum period (13 minutes to 2 hours), to allow reaction to occur. The tissue slice is washed with a TBST washing solution heated to 25 to 60° C. two to five times.

9) A polymer reagent is applied to the tissue slice for 10 minutes to one hour to label bound primary antibodies. The tissue slice is washed with a TBST solution heated to 25 to 60° C. two to five times.

10) A solution containing a coloring agent appropriate for the enzyme (if the enzyme is HRP, diaminobenzidine (DAB), diaminobenzidine nickel (DAB-Ni), or SG provided by Vector, plus hydrogen peroxide) is applied to the tissue slice to allow a coloring reaction to occur. The tissue slice is washed with a TBST solution heated to 25 to 60° C. two to five times.

11) A buffer with a pH adjusted to pH 3 or lower, or pH 9 or higher, e.g., 0.1M glycine hydrochloride buffer with a pH 2.2 is applied to the tissue slice for one minute, the application being repeated three times to allow non-specific enzymatic reaction to occur. The tissue slice is washed with a TBST solution heated to 25 to 60° C. two to five times.

12) A 0.1-1% casein solution is applied to the tissue slice for two to 30 minutes. This is a pretreatment for inhibiting the occurrence of non-specific reaction due to a primary antibody specifically directed to a second antigen (anti-(2nd antigen) primary antibody).

13) An anti-(2nd antigen) primary antibody is applied to the tissue slice at an optimum concentration for an optimum period (13 minutes to 2 hours), to allow reaction to occur. The tissue slice is washed with a TBST washing solution heated to 25 to 60° C. two to five times.

14) A polymer reagent is applied to the tissue slice for 10 minutes to one hour to label bound primary antibodies. The tissue slice is washed with a TBST solution heated to 25 to 60° C. two to five times.

15) A solution containing a coloring agent appropriate for the enzyme (if the enzyme is HRP, one chosen from the following group, but different from the one chosen at step (10), the group comprising DAB, DAB-Ni, and VIP, SG and NovaRed provided by Vector, plus hydrogen peroxide) is applied to the tissue slice to allow a coloring reaction to occur. The tissue slice is washed with a TBST solution heated to 25 to 60° C. two to five times.

16) The tissue is stained with hematoxylin or the like to give a contrastive background, and washed with water one to three times.

17) The tissue slice is removed from the automated immunohistochemical staining system.

18) The tissue slice is sealed with a sealing agent. This serves as a permanent specimen prepared from a fixed, paraffin-embedded tissue slice and undergoing ultra-high sensitive immunohistochemical staining. The specimen serves as a material on which one can observe the target antigens with an optical microscope.

When at steps 10 to 15, substances labeled with strepto-avidin or a fluorescent dye are allowed to deposit on a tissue slice, and at step 15 a solution containing a complex comprising an antibody specifically directed to an antigen and a fluorescent dye is used, after the reaction, a fluorescent dye is applied to the tissue slice to stain nuclei to give a contrastive background, or staining is terminated without counter stain, and the tissue slice is observed with a fluorescent microscope.

When at steps 7 to 10 and 10 to 15, substances labeled with streptoavidin or a fluorescent dye are allowed to deposit on a tissue slice, or at step 15 a solution containing a complex comprising a fluorescent dye and an antibody specifically directed to one kind of primary antibodies is used, omitting step 14, after the reaction, a fluorescent dye is applied to the tissue slice to stain nuclei to give a contrastive background, or the tissue slice is observed with a fluorescent microscope with no counter stain.

Automated operation consists of designing a program for executing the steps 5 to 10 including the use of reaction agents, and setting of reaction periods and washings, and installing the program in an automated immunohistochemical staining system, thereby standardizing the staining operation.

[Details of Reagents]

Details of the used reagents are as follows.

Xylene

Special grade xylene is used.

Ethanol

Special grade ethanol is used.

0.3% Solution of Hydrogen Peroxide in Methanol

A special grade aqueous solution (30%) of hydrogen peroxide is diluted 100 fold with special grade methanol.

0.85% Solution of Sodium Chloride in 0.01M Phosphate Buffer (PBS)

A 28.7 g of sodium phosphate dibasic (Na₂H₂PO₄.12H₂O) and 3.3 g of sodium phosphate monobasic (NaH₂PO₄.2H₂O) are dissolved in 1 L of ion-exchanged water to give a 0.1M phosphate buffer to which 85 g of sodium chloride is added, and the resulting solution is heated in an autoclave until the solute is dissolved completely. The solution is left to be cooled to room temperature. To the solution, a 10N aqueous solution of sodium hydroxide is added to adjust its pH to pH 7.6. This serves as a 10-fold concentrated stock solution. Prior to use, an aliquot extracted from the stock solution is diluted 10 fold with ion-exchanged water.

0.01M Citric Acid Buffer with pH 6.0

A 2.1 g of citric acid monohydrate and 2.94 g of trisodium citrate dihydrate are dissolved in 100 ml of ion-exchanged water while being heated in an autoclave, and to the resulting solution, 10N sodium hydroxide is added to adjust its pH to pH 6.0, to which ion-exchanged water is added to 100 ml. This served as a 10-fold concentrated stock solution. Prior to use, an aliquot extracted from the stock solution is diluted 10 fold with ion-exchanged water. Such 10-fold concentrated stock solutions are available from DakoCytomation and Diatron. For antigen retrieval, a 1 mM EDTA solution with pH 8.0 is used as described in ref. 12(p.114). Such a solution is available from DakoCytomation.

3% Solution of Hydrogen Peroxide in PBS

An aqueous solution of special grade hydrogen peroxide (30% solution) is prepared. A 28.7 g of sodium phosphate dibasic (Na₂H₂PO₄.12H₂O) and 3.3 g of sodium phosphate monobasic (NaH₂PO₄.2H₂O) are dissolved in 1 L of ion-exchanged water to give a 0.1M phosphate buffer to which 85 g of sodium chloride is added, and the resulting solution is heated in an autoclave until the solute is dissolved completely. The solution is left to be cooled to room temperature. To the solution, a 10N aqueous solution of sodium hydroxide is added to adjust its pH to pH 7.6 (PBS). The hydrogen peroxide solution is added to the PBS, and the resulting solution serves as a 10-fold concentrated stock solution. Prior to use, an aliquot extracted from the stock solution is diluted 10 fold with the addition of hydrogen peroxide-free PBS (0.85% solution of sodium chloride in 0.01M phosphate buffer).

0.825 g Sodium Chloride Added 0.05M Tris Buffer Saline (TBS) Containing 0.1% Polyoxyethylene Sorbitan Monolaurate (Tween 20) Heated to about 35° C. used for Washing

A 121.1 g of tris(hydroxymethyl)aminomethane (Sigma) is transferred to 800 ml of ion-exchanged water, and the mixture is heated in an autoclave till complete dissolution. The pH of the resulting solution is adjusted to pH 7.5 with the addition of 1N hydrochloric acid, to which ion-exchanged water is added to 1 L to give a 1M tris buffer solution. A 500 ml of the tris buffer solution and 292.2 g of sodium chloride are transferred to 800 ml of ion-exchanged water, and the mixture is heated in an autoclave till complete dissolution. To the resulting solution, ion-exchanged water is added to 1 L, to give a 5M solution of sodium chloride. A 360 ml of the 5M sodium chloride solution is diluted with ion-exchanged water to 20 L, to give a 0.1% solution of polyoxyethylene sorbitan monolaurate (Tween 20). The solution is transferred to a washing tank of an automated immunohistochemical staining system and heated to about 35° C.

0.25% Casein Solution

A 25 mg of casein (Sigma), 28.7 g of sodium phosphate dibasic (Na₂H₂PO₄.12H₂O) and 3.3 g of sodium phosphate monobasic (NaH₂PO₄.2H₂O) are dissolved in 1 L of ion-exchanged water, to give a 0.1M phosphate buffer to which 85 g of sodium chloride is added, and the mixture is heated in an autoclave till complete dissolution, and left to be cooled to room temperature. To the solution, a 10N aqueous solution of sodium hydroxide is added to adjust its pH to pH 7.6. The resulting solution serves as a 10-fold concentrated stock solution. Prior to use, an aliquot extracted from the stock solution is diluted 10 fold with the addition of casein-free PBS (dissolved in 10 ml of 0.01M PBS).

0.1M Glycine Buffered with Hydrochloric Acid to pH 2.2 (Acidified Glycine)

A 7.5 g of glycine is dissolved in 500 ml of ion-exchanged water, to which concentrated hydrochloric acid is added dropwise to adjust the pH of the solution to pH 2.2, to which ion-exchanged water is added to 1 L.

Polymer Reagent

This is a reagent consisting of a dextran polymer to which a secondary antibody and HRP are directly linked, and is provided by DakoCytomation under the trade name of a ChemMate Envision reagent.

Colored Product of Enzymatic Reaction (Coupling Pigment)

Diaminobenzidine (DAB)

A 20 mg of a tetrachloride of 3,3′-diaminobenzidine (DAB) (Dojin Chemicals) is dissolved in 100 ml of 0.05M tris hydrochloric acid buffer (pH 7.6), to which 20 μl of pure hydrogen peroxide is added, to be used. The same liquid reagent is available from DakoCytomation under the trade name of K3466. Vector provides the reagent in a DAB substrate kit (SK-4100). The 0.05M tris hydrochloric acid buffer (pH 7.6) is obtained as follows. A 121.1 g of tris(hydroxymethl)aminomethane (Sigma) is dissolved in 800 ml of ion-exchanged water, and the mixture is heated in an autoclave till complete dissolution. To the solution, 1N hydrochloric acid is added to adjust its pH to pH 7.6. To the solution, ion-exchanged water is added to 1 L to give a 1M tris solution. A 500 ml of the 1M tris solution and 292.2 g of sodium chloride are dissolved in 800 ml of ion-exchanged water, and the mixture is heated in an autoclave till complete dissolution. To the solution, ion-exchanged water is added to 1 L, to give a 5M sodium chloride solution. A 360 ml of the 5M sodium chloride solution is diluted with ion-exchanged water to 20 L.

Diaminobenzidine Nickel (DAB-Ni)

A 20 mg of DAB, and 40 mg of nickel sulfide (NiSO₄.(NH₄)₂SO₄.6H₂O) are dissolved in 100 ml of 0.05M tris hydrochloric acid buffer (pH 7.6), to which 7 μl of pure hydrogen peroxide is added, and the solution is filtered to be used. Vector provides the same reagent in a DAB substrate kit (SK-4100).

Aminoethylcarbarzole (AEC)

3-Amino-9-ethylcarbazole (Sigma) is dissolved in dimethylsulfoxide (DMSO) to give a 0.4% solution. This solution serves as a stock solution. A 5 ml of an aliquot is mixed with 95 ml of 0.1M acetic acid buffer with pH 5.0 to which 20 μl of pure hydrogen peroxide is added, and the resulting solution is filtered to be used. The 0.1M acetic acid buffer with pH 5.0 is obtained as follows. A 11.5 ml of 99% acetic acid is dissolved in ion-exchanged water to 1 L. A 14.8 ml of the resulting solution, and 16.4 g of sodium acetate or 27.2 g of sodium acetate trihydrate are dissolved in ion-exchanged water to 1 L, to which 35.2 ml of 0.2M sodium acetate is added, and the resulting solution is diluted with ion-exchanged water to 100 ml to give the desired acetic acid buffer. The same buffer is available in a kit provided by DakoCytomation.

VIP, SG, NovaRED

These coupling pigments developing a color through the reaction with HRP are available from Vector under the trade name of SK-4600, SK-4700 and SK-4800.

Hematoxylin Solution and Others

The nuclei or cytoplasm of cells contained in an immunohisto-chemically stained tissue slice is further stained with a dye exhibiting a color different from that of a coupling pigment to give a contrastive background. Generally, for this purpose, a hematoxylin solution is used. DakoCytomation provides a DakoChemMate hematoxylin reagent which serves as a suitable dye when used in combination with an automated immunohistochemical staining system.

Sealing in Correspondence with Foregoing Processes

Sealing of a stained tissue slice should be made in correspondence with the foregoing staining process. If diaminobenzidine is used as a coupling pigment, the tissue slice should be passed through serial ethanol solutions to be dehydrated, and sealed with a plastic sealing agent (Eukitt) to provide a permanently preservable specimen. If AEC is used, an Ultramount reagent provided by DakoCytomation is applied to the tissue slice, and heated to 70° C. to be solidified, to provide a permanently preservable specimen. If one of the coupling agents provided by Vector is used, the tissue slice is dried by being exposed to a blow of cold air, sealed with VectaMount or a sealing agent, and dried to be solidified, to provide a permanently preservable specimen.

Next, procedures actually performed for inhibiting non-specific reactions, and tests for confirming the effects of the buffer will be described.

Steps 1 to 10 (excepting step 7) and steps 16 to 18 are actually performed. As a buffer with pH 3 or lower, or pH 9 or higher required for step 11, 0.1M glycine buffered with hydrochloric acid to pH 2.2 (acidified glycine) is used. Between steps 9 and 10, is inserted step 11 in which the application time of the acidified glycine is varied (Experiment A). In a separate run, step 11 in which the application time of the acidified glycine is similarly varied is inserted between steps 8 and 9 (Experiment B). The test tissue slice is a fixed, paraffin-embedded slice obtained from a human tonsilla fixed in 10% formalin. Antigen retrieval is performed by immersing the tissue slice in 0.01M citric acid buffer with pH 6.0 and transferring the system to an autoclave to be heated. An anti CD 68 antibody specifically directed to dendritic cells distributed in lymphatic nodules is used. The polymer reagent required for step 9 is a ChemMate Envision reagent provided by Dako. The enzyme required for step 10 is HRP in combination with DAB as a coupling pigment. In step 18, the tissue slice is dehydrated with ethanol, soaked with xylene, and sealed with a plastic sealing agent (Eukitt).

Experiment A is designed for testing the effect of the acidified glycine in removing a complex (polymer reagent) comprising the primary and secondary antibodies and enzyme. Experiment B is for testing the effect of the acidified glycine in removing the primary antibody. Step 7 is omitted in Experiment A and B, but carried out in Experiment B#.

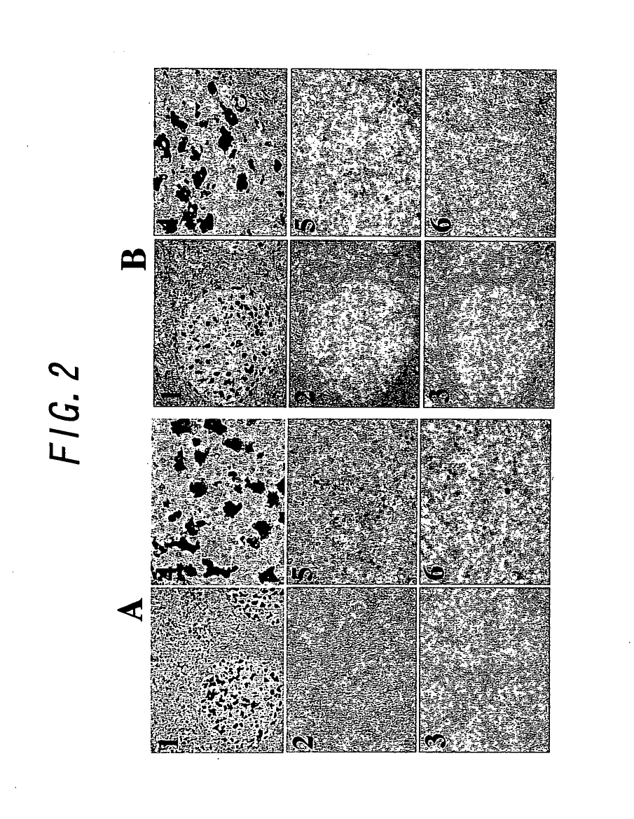

As shown in Table 1, in Experiment A, no DAB reaction is present whether the application time of the acidified glycine is varied to 2, 7, 17, 37 and 77 minutes. In Experiment B, the tissue slice exhibited a faint color even when the acidified glycine is applied for 77 minutes, which indicates that non-specifice reaction products are not yet removed. Since the non-specific reaction products are likely to result from primary antibodies bound to non-specific reactive substances, treatment with a 0.25% casein solution as described above in step 7 is added (Experiment B#). Then, it is found that, even when the casein solution is applied for only one minute, non-specific primary antibody products are removed. In FIG. 2, panel A indicates the photomicrographs of tissue slices stained in Experiment B while panel B those of tissue slices stained in Experiment B#. In FIG. 2, the first row of photographs (A1, A4, B1 and B4) indicate the tissues to which no acidified glycine is applied, the second row of photographs (A2, A5, B2 and B5) those to which the acidified glycine is applied for 17 minutes, and the third row of photographs (A3, A6, B3 and B6) those to which the acidified glycine is applied for 37 minutes. Comparison between tissue images AS and B5, and A6 and B6 indicates that addition of 0.25% casein treatment as in step 7 removes non-specific reactions in tissue images B5 and B6 in contrast with tissue images A5 and A6.

In Example 1, it is shown that when the acidified glycine is used as the buffer with pH adjusted to pH 3 or lower, or pH 9 or higher as described above in step 11, it dissociates the binding of antibodies to antigens even it is applied to the test tissue slice for only one minute. This result suggests that, if a tissue slice is treated, in step 11, with 0.1M glycine buffered with hydrochloric acid to pH 2.2 for one minute three times, so that the production of non-specific primary antibody products is inhibited, it will be possible to remove primary antibodies together with their marker non-specifically bound to the fixed, paraffin-embedded tissue slice. Table 1 shows the evaluation results regarding non-specific reactive products remaining in tissue slices after the acidified glycine treatment.

TABLE 1

| | Glycine treatment time (min) | | | | | | | | | | |
|---|---|---|---|---|---|---|---|---|---|---|---|
| | 1 | 2 | 3 | 4 | 6 | 7 | 8 | 10 | 17 | 37 | 77 |
| Experiment A | | − | | | | − | | | − | − | − |
| Experiment B | | + | | | | +/− | | | +/− | +/− | +/− |
| Experiment B # | − | − | − | − | − | − | − | − | − | − | − |

#: Protein block for inhibiting non-specific reaction is performed before introduction of primary antibodies. Evaluation of non-specific reactive products remaining in tissue slices after glycine treatment.
(+: Positive residue, +/−: faint residue, −: no residue)

[Procedures and Reagents Performed and Used in Example 1]
0.1M Glycine Buffered with Hydrochloric acid to pH 2.2 (Acidified Glycine)

A 7.5 g of glycine is dissolved in 500 ml of ion-exchanged water, to which concentrated hydrochloric acid is added dropwise to adjust the pH of the solution to pH 2.2, to which ion-exchanged water is added to 1 L.

Antigen Retrieval

A fixed tissue slice removed of paraffin is immersed in 0.01M citric acid buffer with pH 6.0, and heated in an autoclave.

0.01M Citric Acid Buffer with pH 6.0

A 2.1 g of citric acid monohydrate and 2.94 g of trisodium citrate dihydrate are dissolved in 100 ml of ion-exchanged water while being heated in an autoclave, and to the resulting solution, 10N sodium hydroxide is added to adjust its pH to pH 6.0, to which ion-exchanged water is added to 100 ml. This serves as a 10-fold concentrated stock solution. Prior to use, an aliquot extracted from the stock solution is diluted 10 fold with ion-exchanged water. Such 10-fold concentrated stock solutions are available from DakoCytomation and Diatron.

Anti CD68 Antibody

The used antibody is a mouse anti human CD68 macrophage antibody (PG-M1, M0876) available from DakoCytomation. The product provided by DakoCytomation is diluted 50 fold by the addition of DakoChemMate antibody diluent (S2022) prior to use.

Polymer Reagent

This is a reagent consisting of a dextran polymer to which a secondary antibody and HRP are directly linked, and is provided by DakoCytomation under the trade name of a ChemMate Envision reagent.

Colored Product of Enzymatic Reaction (Coupling Pigment)

Diaminobenzidine (DAB)

A liquid reagent provided by DakoCytomation under the trade name of K3466 is used.

### Example 2

Next, the effect of the acidified glycine on antigens is examined.

Steps 1 to 6 and steps 11 to 18 are performed. A fixed slice obtained from the human tonsilla is used. Antigen retrieval is performed by immersing the tissue slice in 0.01M citric acid buffer with pH 6.0 and transferring the system to an autoclave to be heated. An antibody specifically directed to CD 68 or Ki-67 antigen is used as a primary antibody. The effect of the acidified glycine as described in step 11 above on the antigens is studied. The polymer reagent as described in step 14 above is a ChemMate Envision reagent provided by Dako. The enzyme as described step 15 above is HRP in combination with DAB as a coupling pigment. In step 18, the tissue slice is dehydrated with ethanol, soaked with xylene, and sealed with a plastic sealing agent (Eukitt).

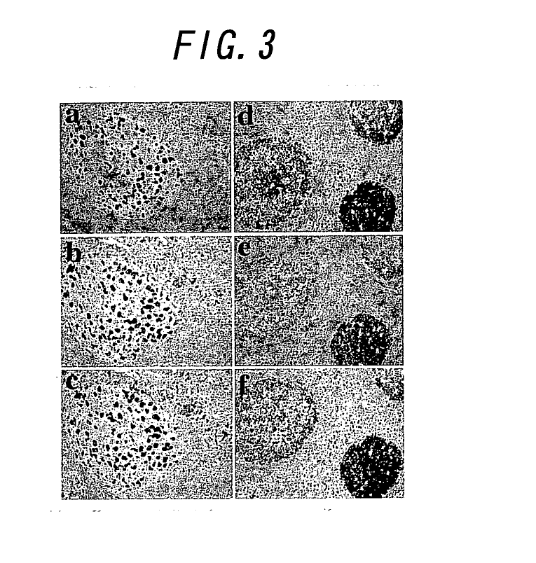

The results are shown in FIG. 3. In FIG. 3, the left column of photographs a, b and c represent tissue slices treated with the anti CD68 antibody while the right column of photographs d, e and f those treated with the anti Ki-67 antibody. The rows of photographs are arranged according to the treatment time of the acidified glycine: the first row consisting of photographs a and d represents tissue slices treated with phosphate buffer with pH 7.4, the second row of photographs b and e those treated with the acidified glycine for seven minutes, and the third row of photographs c and f those treated with the acidified glycine for 17 minutes. The tissue slices treated with the anti CD68 antibody do not show any change even when the acidified glycine is applied even up to 77 minutes. On the other hand, with regard to the tissue slices treated with the anti Ki67 antibody, non-specific antibody products are found to be slightly masked anew after the acidified glycine is applied for seven minutes. This suggests that the detection of antigen Ki-67 should occur before the removal of non-specific antibody products due to the acidified glycine.

This suggests further that, in the inventive multiplicated immuno-histochemistry, it is necessary, prior to execution of the method, to determine, for a given target antigen, which is better for the detection of that antigen between the two cases: in one case, the detection in question occurs before the removal of non-specific antibody products, and in the other the detection in question occurs after the removal of non-specific antibody products.

[Procedures and Reagents Performed and used in Example 2]

Antigen Retrieval Treatment

A fixed tissue slice removed of paraffin is treated with an enzyme such as trypsin, or immersed in a buffer such as 0.0M citric acid buffer with pH 6.0, and heated. Heating may take place in a microwave heater, autoclave, pressure cooker, warm water bath, etc. The enzymatic and heating treatments may be changed according to the type of target antigens.

0.01M Citric Acid Buffer with pH 6.0

A 2.1 g of citric acid monohydrate and 2.94 g of trisodium citrate dihydrate are dissolved in 100 ml of ion-exchanged water while being heated in an autoclave, and to the resulting solution, 10N sodium hydroxide is added to adjust its pH to pH 6.0, to which ion-exchanged water is added to 100 ml. This serves as a 10-fold concentrated stock solution. Prior to use, an aliquot extracted from the stock solution is diluted 10 fold with ion-exchanged water. Such 10-fold concentrated stock solutions are available from DakoCytomation and Diatron.

Anti CD68 Antibody

The used antibody is a mouse anti human CD68 macrophage antibody (PG-M1, M0876) available from DakoCytomation. The product provided by DakoCytomation is diluted 50 fold by the addition of DakoChemMate antibody diluent (S2022) prior to use.

Anti Ki-67 Antibody

The used antibody is a mouse anti Ki-67 antibody (MIB-1, M7240) available from DakoCytomation. The product provided by DakoCytomation is diluted 50 fold by the addition of DakoChemMate antibody diluent (S2022) prior to use.

0.1M Glycine Buffered with Hydrochloric Acid to pH 2.2 (Acidified Glycine)

A 7.5 g of glycine is dissolved in 500 ml of ion-exchanged water, to which concentrated hydrochloric acid is added dropwise to adjust the pH of the solution to pH 2.2, to which ion-exchanged water is added to 1 L.

Polymer Reagent

This is a reagent consisting of a dextran polymer to which a secondary antibody and HRP are directly linked, and is provided by DakoCytomation under the trade name of a ChemMate Envision reagent.

Colored Product of Enzymatic Reaction (Coupling Pigment)

Diaminobenzidine (DAB)

A liquid reagent provided by DakoCytomation under the trade name of K3466 is used.

### Example 3

Steps 1 to 6 and steps 12 to 18 are performed. A fixed slice obtained from the human tonsilla is used. Antigen retrieval is performed by immersing the tissue slice in 0.01M citric acid buffer with pH 6.0 and transferring the system to an autoclave to be heated. An antibody specifically directed to CD 68 antigen is used as a primary antibody. The effect of the acidified glycine as described in step 11 above on the antigens is studied. A ChemMate Envision reagent provided by Dako is used as a polymer reagent to study. The followings are studied.

Coupling pigments utilized in combination with the reaction between HRP and hydrogen peroxide, and marketed under the trade name of DAB and AEC by DakoCytomation, and DAN-Ni, NovaRed, VIP, TMB, SG and TMB by Vecor are examined in combination with methods for sealing the tissue slice.

The post-staining treatments are as follows. In a normal treatment, a tissue slice is dehydrated with ethanol, soaked with xylene, and sealed with a plastic sealing agent. In a Ultramount method, Ultramount provided by DakoCytomation is applied dropwise on a tissue slice which is then heated to 70° C. to be dried. In a VectaMount method, VectaMount provided by Vector is applied dropwise to a tissue slice which is then covered with a slide glass, to be left for drying.

The tissue slice is converted with a microscope to a digital color image, the color of a positively stained spot is decomposed by means of Photoshop into three components corresponding to R, G and B channels, the average is determined for each channel, and the averages corresponding to R, G and B channels are taken as the R, G and B components, respectively.

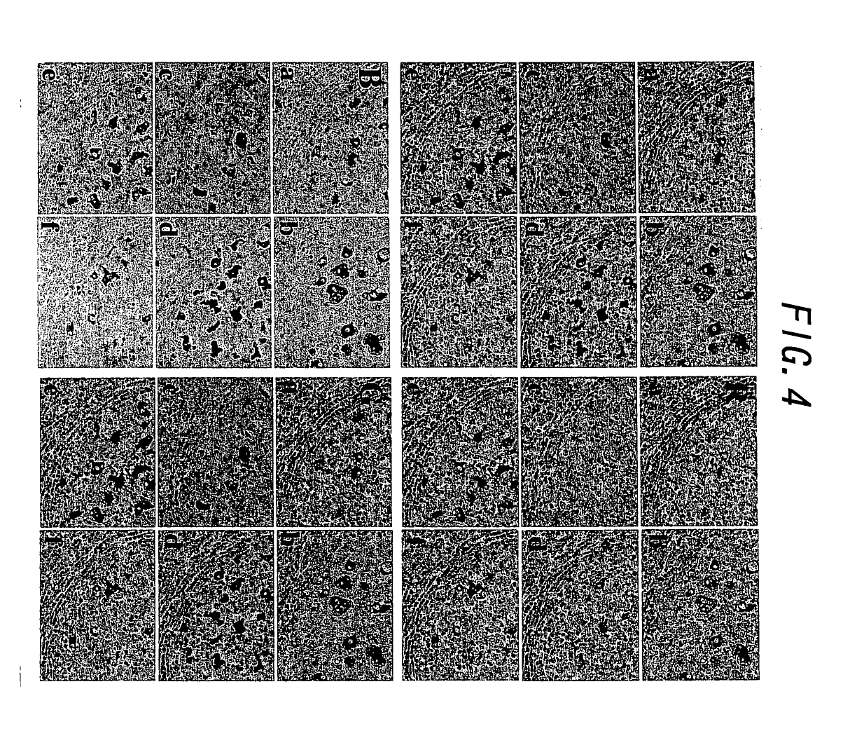

Utilizable pigments are restricted by the sealing method. FIG. 4 show the images of tissue slices represented in full color (or gray scale) or in R, G or B components. Tables 2 to 4 list the magnitudes of R, G and B components of those tissue images. FIG. 4A represents the full-color images of tissue slices when the combination of pigments with sealing methods is varied: photograph a represents the image of a tissue stained with DAB and sealed by the normal method (see Table 2); photograph b that stained with DAB-Ni and sealed by the normal method (see Table 2); photograph c that stained with AEC and sealed by the Ultramount method (see Table 3); photograph d that stained with NovaRed and sealed by the VectorMount method (see Table 4); photograph e that stained with VIP and sealed by the VectaMount method (see Table 4); and photograph f that stained with SG and sealed by the VectaMount method (see Table 4). When the R, G and B components of those images are compared, in image a the G and B components are comparatively large; in image b the R, G and B components are equally large; in image c the G and B components are comparatively large as in the image stained with DAB; in images d and e, the R, G and B components are large; and in image f the R, G and B components are large. R-component images stained with DAB and AEC can be distinguished from corresponding images stained with NovaRed and VIP. Table 2 shows that the images of tissues stained with different pigments exhibit characteristic profiles of R, G and B components. When the normal method is used for sealing, DAB, DAB-Ni and SG are usable. When such tissues are further treated with glycine, their images are found usable, although they increased in the R component with a reduction in the B component. Thus, those combinations are found usable for the detection of multiple antigens. When the Ultramount method is used for sealing, DAB, DAB-Ni AEC and SG are usable. When the Vectamount method is used for sealing, DAB, DAB-Ni, NovaRed VIP and SG are usable.

SG stained antigens weakly as compared with other pigments, but is resistant to other processings such as sealing or glycine treatment. Thus, SG can be used, in multiplicated staining involving a highly active antigen and a weakly active antigen, as a pigment staining the highly active antigen.

TMB's deposition after reaction is faint, and is decolorized after sealing. Tables 2 to 4 list the magnitudes of R, G and B components of tissue images which are stained with coupling pigments activated as a result of the reaction between HRP and hydrogen peroxide and sealed by the various sealing methods.

TABLE 2

| Normal method | R | G | B |
|---|---|---|---|
| DAB(a) | 85 | 51 | 48 |
| DAB #1 | 97 | 46 | 36 |
| DAB-Ni (b) | 97 | 67 | 66 |
| DAB-Ni #1 | 105 | 66 | 57 |
| SG | 37 | 42 | 74 |
| SG #1 | 34 | 41 | 69 |
| Hematoxylin | 138 | 144 | 189 |

#the pigment preceded by glycine treatment

TABLE 3

| Ultramount method | R | G | B |
|---|---|---|---|
| DAB | 107 | 67 | 62 |
| DAB-Ni | 74 | 46 | 44 |
| AEC(c) | 141 | 55 | 52 |
| Hematoxylin | 148 | 153 | 192 |

TABLE 4

| VectaMount method | R | G | B |
|---|---|---|---|
| DAB | 89 | 40 | 30 |
| DAB-Ni | 77 | 51 | 52 |
| NovaRED (d) | 113 | 45 | 39 |
| VIP (e) | 57 | 26 | 68 |
| SG (f) | 30 | 36 | 65 |
| Hematoxylin | 164 | 166 | 190 |

[Procedures and Reagents Performed and Used in Example 3]

Antigen Retrieval

A fixed tissue slice removed of paraffin is immersed in 0.0M citric acid buffer with pH 6.0, and heated. Heating may take place in an autoclave.

0.01M Citric Acid Buffer with pH 6.0

A 2.1 g of citric acid monohydrate and 2.94 g of trisodium citrate dihydrate are dissolved in 100 ml of ion-exchanged water while being heated in an autoclave, and to the resulting solution, 10N sodium hydroxide is added to adjust its pH to pH 6.0, to which ion-exchanged water is added to 100 ml. This served as a 10-fold concentrated stock solution. Prior to use, an aliquot extracted from the stock solution is diluted 10 fold with ion-exchanged water. Such 10-fold concentrated stock solutions are available from DakoCytomation and Diatron. Alternatively, antigen retrieval may be performed by using 1 mM EDTA with pH 8.0 in the manner as described in ref. 12 (p. 114). Such an EDTA solution is available from DakoCytomation.

Anti CD68 Antibody

The used antibody is a mouse anti human CD68 macrophage antibody (PG-M1, M0876) available from DakoCytomation. The product provided by DakoCytomation is diluted 50 fold by the addition of DakoChemMate antibody diluent (S2022) prior to use.

Polymer Reagent

This is a reagent consisting of a dextran polymer to which a secondary antibody and HRP are directly linked, and is provided by DakoCytomation under the trade name of a ChemMate Envision reagent.

Colored Product of Enzymatic Reaction (Coupling Pigment) Diaminobenzidine (DAB)

A liquid reagent provided by DakoCytomation under the trade name of K3466 is used.

Diaminobenzidine Nickel (DAB-Ni)

A 20 mg of DAB, and 40 mg of nickel sulfide (NiSO₄.(NH₄)₂SO₄.6H₂O) are dissolved in 100 ml of 0.05M tris hydrochloric acid buffer (pH 7.6), to which 7 μl of pure hydrogen peroxide is added, and the solution is filtered to be used. Vector provides the same reagent in a DAB substrate kit (SK-4100).

Aminoethylcarbazole (AEC)

3-Aminoethyl-9-ethylcarbazole (Sigma) is dissolved in dimethyl-sulfoxide (DMSO) to give a 0.4% solution. This solution served as a stock solution. A 5 ml of an aliquot is mixed with 95 ml of 0.1M acetic acid buffer with pH 5.0 to which 20 μl of pure hydrogen peroxide is added, and the resulting solution is filtered to be used. The 0.1M acetic acid buffer with pH 5.0 is obtained as follows. A 11.5 ml of 99% acetic acid is dissolved in ion-exchanged water to 1 L. A 14.8 ml of the resulting solution, and 16.4 g of sodium acetate or 27.2 g of sodium acetate trihydrate are dissolved in ion-exchanged water to 1 L, to which 35.2 ml of 0.2M sodium acetate is added, and the resulting solution is diluted with ion-exchanged water to 100 ml to give the desired acetic acid buffer. The same buffer is available in a kit provided by DakoCytomation.

VIP, SG, NovaRED and TMB

These coupling pigments developing a color through the reaction with HRP are available from Vector under the trade name of SK-4600, SK-4700, SK-4800 and SK-4400.

Sealing for Permanent Specimens

After staining, tissue slices are passed through serial ethanol solutions to be dehydrated, soaked with xylene, and sealed with a plastic sealing agent (Eukitt). This is a commonly used sealing method and is called a normal method.

Ultramount Method

A Ultramount medium (S1964) provided by DakoCytomation or a medium used for the preservation of permanently preservable specimens is applied dropwise to tissue slices to cover their entire surfaces, and then the slices are heated in an oven to be solidified.

VectaMout Method

VectaMount (H-5000) provided by Vector is applied dropwise so that it covered the entire surface of a fixed tissue slice, and then the assembly is left so that the medium is dried spontaneously to be solidified.

Photoshop

This is a computer software product for processing image provided by Adobe. In this experiment, Photoshop 3.0J is used. Colored images of tissue slices on colored photomicrographs are fed to a computer in which Photoshop is installed, and decomposed into R, G and B components, and into a CMYK gray component. To determine R, G and B components of a colored image, it is necessary to obtain a reversed-color image of that original colored image. For this purpose, a software product or N1H-image is used.

N1H-Image

This is an image analysis software product. It is possible using this software to obtain, for a given colored image, its average gray scale and area (in terms of the number of pixels).

### Example 4

As an illustrative example of duplicate immunohistochemical staining for two antigens which are localized at the same sites, two antigens CD68 and Fascin are stained. Coupling pigments activated via the reaction between HRP and hydrogen peroxide are used for the detection of the two antigens, and combinations of pigments are evaluated. As it is, since the actual staining involves executing a counter stain of nuclei using hematoxylin as shown in Table 2, triplicate staining is performed. An anti CD68 antibody is specifically directed to monocytes and histiocytes while an anti Fascin antibody is specifically directed to dendritic cells. A fixed tissue slice obtained from the human tonsil is used. Antigen retrieval is achieved by immersing the tissue slice in EDTA, and heating the tissue in an autoclave. For the detection of bound primary antibodies, a polymer reagent or ChemMate Envision is used.

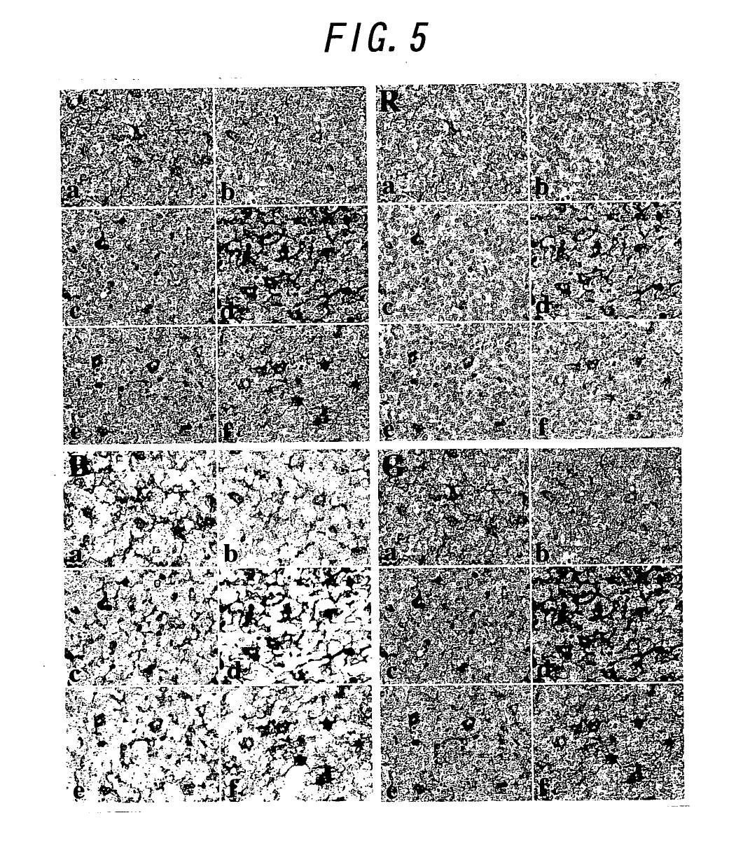

Such doubly stained tissue slices as described above are shown in FIG. 5. The combinations of coupling pigments are DAB-Ni and DAB for photograph a, DAB-Ni and AEC for photograph b, DAB and AEC for photograph c, DAB and VIP for photograph d, SG and AEC for photograph e, and SG and NovaRed for photograph f. From FIG. 5, it is suggested that the following combinations of pigments are usable, that is, DAB and AEC, DAB and VIP, SG and AEC, and SG and NovaRed. It is also found that some combinations are effective for detecting the two antigens located on the same site.

Table 2 shows the combination of DAB-Ni and DAB may not be appropriate for discriminating between the two antigens: combination of DAB-Ni with glycine treatment increases the R component which may serve as a distinctive feature to discriminate between the two antigens, but combination of DAB-Ni with glycine treatment reduces the B component which causes the B components of the two antigens in the same tissue slice to look similar as shown in FIG. 5a.

The same observation also applies to the combination of DAB-Ni and AEC: combination of DAB-Ni with glycine treatment increases the R component which causes the R components of the two antigens in the same tissue slice to look similar as shown in FIG. 5b.

As far as judged from the RGB images as well as gray scale images, combination of DAB and VIP is best for discriminating between the two antigens, especially when, for a given image, its B component DAB image is compared with its R component VIP image.

[Procedures and Reagents Performed and Used in Example 4]

Antigen Retrieval

A fixed tissue slice removed of paraffin is treated with an enzyme such as trypsin, or immersed in a buffer such as 0.0M citric acid buffer with pH 6.0, and heated. Heating may take place in a microwave heater, autoclave, pressure cooker, warm water bath, etc. The enzymatic and heating treatments may be changed according to the type of target antigens.

1 mM to 0.01M EDTA with pH 8.0

A 0.37 g of EDTA disodium (Dojin Chemicals) is added to 1 L of ion-exchanged water, to which 1N sodium hyroxide is added to adjust its pH to pH 8.0. Such an EDTA solution is available from DakoCytomation.

Anti CD68 Antibody

The used antibody is a mouse anti human CD68 macrophage antibody (PG-M1, M0876) available from DakoCytomation. The product provided by DakoCytomation is diluted 50 fold by the addition of DakoChemMate antibody diluent (S2022) prior to use.

Anti Fascin Antibody

The used antibody is a mouse anti human Fascin protein antibody

K-2, M3567) available from DakoCytomation. The product provided by DakoCytomation is diluted 50 fold by the addition of DakoChemMate antibody diluent (S2022) prior to use.

Polymer Reagent

This is a reagent consisting of a dextran polymer to which a secondary antibody and HRP are directly linked, and is provided by DakoCytomation under the trade name of a ChemMate Envision reagent.

Colored Product of Enzymatic Reaction (Coupling Pigment)

Diaminobenzidine (DAB)

A liquid reagent provided by DakoCytomation under the trade name of K3466 is used.

Diaminobenzidine Nickel (DAB-Ni)

A 20 mg of DAB, and 40 mg of nickel sulfide (NiSO₄.(NH₄)₂SO₄.6H₂O) are dissolved in 100 ml of 0.05M tris hydrochloric acid buffer (pH 7.6), to which 7 μl of pure hydrogen peroxide is added, and the solution is filtered to be used. Vector provides the same reagent in a DAB substrate kit (SK-4100).

Aminoethylcarbazole (AEC)

3-Aminoethyl-9-ethylcarbazole (Sigma) is dissolved in dimethyl-sulfoxide (DMSO) to give a 0.4% solution. This solution serves as a stock solution. A 5 ml of an aliquot is mixed with 95 ml of 0.1M acetic acid buffer with pH 5.0 to which 20 μl of pure hydrogen peroxide is added, and the resulting solution is filtered to be used. The 0.1M acetic acid buffer with pH 5.0 is obtained as follows. A 11.5 ml of 99% acetic acid is dissolved in ion-exchanged water to 1 L. A 14.8 ml of the resulting solution, and 16.4 g of sodium acetate or 27.2 g of sodium acetate trihydrate are dissolved in ion-exchanged water to 1 L, to which 35.2 ml of 0.2M sodium acetate is added, and the resulting solution is diluted with ion-exchanged water to 100 ml to give the desired acetic acid buffer. The same buffer is available in a kit provided by DakoCytomation.

VIP, SG, NovaRED and TMB

These coupling pigments developing a color through the reaction with HRP are available in a kit from Vector under the trade name of SK-4600, SK-4700 and SK-4800.

Gray Scale Image

An image whose shades of gray are expressed, for each of its constitutive pixels, by one of 256 steps between 0 and 255. A pixel is a constitutive element of an image that has the smallest area. A full color image, when decomposed by Photoshop capable of RGB channel decomposition, is decomposed into R, G and B component images and into a gray scale image.

### Example 5

As an illustrative example of duplicate immunohistochemical staining for two antigens which are localized partially differently, two antigens CD3 and CD5 are stained. CD3 is an antigen present on T cells while CD5 is present on T cells and part of B cells. The test tissue slice is a fixed, paraffin-embedded slice obtained from a human tonsilla fixed in 10% formalin. Antigen retrieval is performed by immersing the tissue slice in 0.01M citric acid buffer with pH 6.0 and transferring the system to an autoclave to be heated. For the detection of bound primary antibodies, a polymer reagent or ChemMate Envision is used. Coupling pigments used in combination with HRP are DAB for the detection of CD3, and VIP for the detection of CD5.

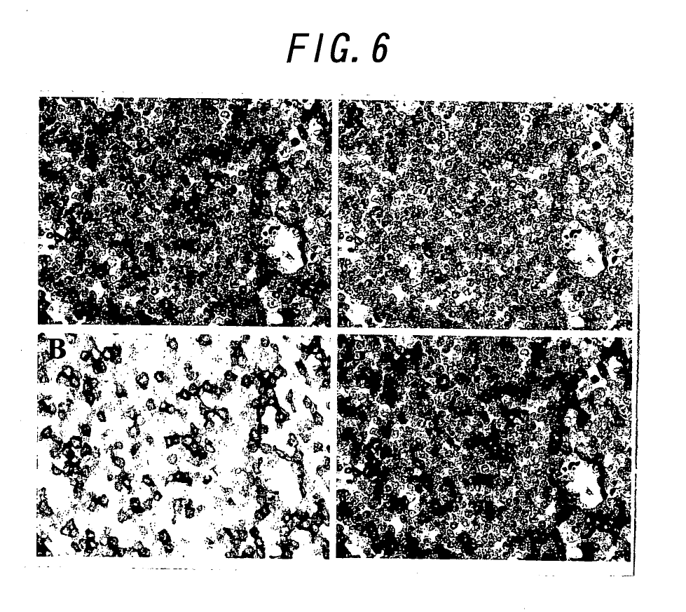

FIG. 6 shows the photographic images of a tissue around the mantle of a lymphoid follicle. For each photographic image, CD3 positive, CD5 positive T cells are more densely distributed on the left side, and in the R-component image of the tissue they stain positively. The right side of the figure is towards a germinal center. The center of the figure is at a boundary between the mantle and the periphery of the follicle where CD3 negative, CD5 positive B cells are present. These CD3 negative, CD5 positive B cells stain positively in the R component image of the tissue. These B cells appear, when stained with DAB, only in the B and G component images of the tissue, while the same cells are appear, when stained with VIP, in all the B, G and R component images of the tissue.

[Procedures and Reagents Performed and Used in Example 5]

Antigen Retrieval

A fixed tissue slice removed of paraffin is treated with an enzyme such as trypsin, or immersed in a buffer such as 0.0M citric acid buffer with pH 6.0, and heated. Heating may take place in a microwave heater, autoclave, pressure cooker, warm water bath, etc. The enzymatic and heating treatments may be changed according to the type of target antigens.

0.01M Citric Acid Buffer

A 2.1 g of citric acid monohydrate and 2.94 g of trisodium citrate dihydrate are dissolved in 100 ml of ion-exchanged water while being heated in an autoclave, and to the resulting solution, 10N sodium hydroxide is added to adjust its pH to pH 6.0, to which ion-exchanged water is added to 100 ml. This served as a 10-fold concentrated stock solution. Prior to use, an aliquot extracted from the stock solution is diluted 10 fold with ion-exchanged water.

Anti CD3 Antibody

The used antibody is a mouse anti human CD3 antibody (NCL-CD3-PS1) available from Novocastra. The product provided by Novocastra is diluted 100 fold by the addition of DakoChemMate antibody diluent (S2022) prior to use.

Anti CD5 Antibody

The used antibody is a mouse anti human CD5 antibody (NCL-CD5-4C7) available from Novocastra. The product provided by Novocastra is diluted 100 fold by the addition of DakoChemMate antibody diluent (S2022) prior to use.

Polymer Reagent

This is a reagent consisting of a dextran polymer to which a secondary antibody and HRP are directly linked, and is provided by DakoCytomation under the trade name of a ChemMate Envision reagent.

Diaminobenzidine (DAB)

A liquid reagent provided by DakoCytomation under the trade name of K3466 is used.

VIP

This coupling pigment developing a color through the reaction with HRP is available in a kit from Vector under the trade name of SK-4600.

### Example 6

As an illustrative example of duplicate immunohistochemical staining for two antigens which are localized differently, two antigens CD3 and CD79a are stained. CD79a is an antigen present on B cells. Antigen retrieval is performed by immersing a tissue slice in 0.01M citric acid buffer with pH 6.0 and transferring the system to an autoclave to be heated. The tissue slice is mounted on an automated immunohistochemical staining system and the enzymatic reaction is allowed to occur at 35° C. for five minutes with the activity of the enzyme being reduced to 1/10 of the normal level according to a protocol installed in the system. The test tissue slice is a fixed, paraffin-embedded slice obtained from a human tonsil fixed in 10% buffered formalin. Antigen retrieval is performed by immersing the tissue slice in 0.01M citric acid buffer with pH 6.0 and transferring the system to an autoclave to be heated. For the detection of bound primary antibodies, a polymer reagent or ChemMate Envision is used. Coupling pigments used in combination with HRP are DAB for the detection of CD3, and VIP for the detection of CD79a.

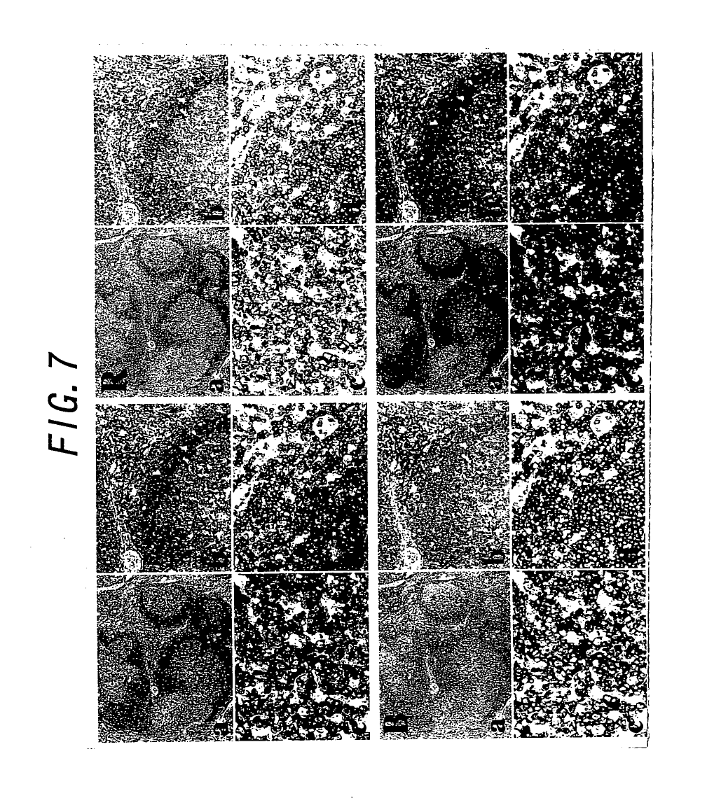

FIG. 7 shows the photographic images of the stained tissue. In FIG. 7, photographs a and c are low and high magnification images of a germinal center of a lymph node, respectively, while photographs b and d are low and high magnification images of a peripheral area of a follicle apart from the mantle. T cells stained with DAB develop a brown color, and stain positively in the B component image. B cells develop a purple color, and stain positively in the R component image. The inventive immunohistochemical staining clearly distinguishes T cells and B cells.

[Procedures and Reagents Performed and Used in Example 6]

Antigen Retrieval

A fixed tissue slice removed of paraffin is treated with an enzyme such as trypsin, or immersed in a buffer such as 0.0M citric acid buffer with pH 6.0, and heated. Heating may take place in a microwave heater, autoclave, pressure cooker, warm water bath, etc. The enzymatic and heating treatments may be changed according to the type of target antigens.

0.01M Citric Acid Buffer with pH 6.0

A 2.1 g of citric acid monohydrate and 2.94 g of trisodium citrate dihydrate are dissolved in 100 ml of ion-exchanged water while being heated in an autoclave, and to the resulting solution, 10N sodium hydroxide is added to adjust its pH to pH 6.0, to which ion-exchanged water is added to 100 ml. This served as a 10-fold concentrated stock solution. Prior to use, an aliquot extracted from the stock solution is diluted 10 fold with ion-exchanged water.

Enzymatic Treatment

A table to trypsin (T7168 Tablet, Sigma) is dissolved in 1 ml of ion-exchanged water, and the resulting solution is used as a reactive solution.

Pronase

A 100 mg of pronase (DakoCytomation) is dissolved in 10 ml of 0.05M tris hydrochloric acid buffer with pH 7.0 containing sodium chloride at 0.1 M/L. The resulting solution is diluted 20 fold by the addition of 0.05M tris hydrochloric acid buffer with pH 7.2 containing sodium chloride at 0.1 M/L.

Proteinase K

A 40 μl of proteinase K concentrated solution (S3004, DakoCytomation) is added to 2 ml of 0.05M tris hydrochloric acid buffer with pH 7.5-7.7.

Anti CD3 Antibody

The used antibody is a mouse anti human CD3 antibody (NCL-CD3-PS1) available from Novocastra. The product provided by Novocastra is diluted 100 fold by the addition of DakoChemMate antibody diluent (S2022) prior to use.

Anti CD79a Antibody

The used antibody is a mouse anti human CD79a antibody (CD79a, B set, JCB117, M7050) available from Novocastra. The product provided by Novocastra is diluted 200 fold by the addition of DakoChemMate antibody diluent (S2022) prior to use.

Polymer Reagent

This is a reagent consisting of a dextran polymer to which a secondary antibody and HRP are directly linked, and is provided by DakoCytomation under the trade name of a ChemMate Envision reagent.

Diaminobenzidine (DAB)

A liquid reagent provided by DakoCytomation under the trade name of K3466 is used.

VIP

This coupling pigment developing a color through the reaction with HRP is available in a kit from Vector under the trade name of SK-4600.

### Example 7

As an illustrative example of duplicate immunohistochemical staining for two antigens which are localized at the same sites, two antigens p53 protein and Ki-67 are stained. The p53 protein is a protein coded by a tumor suppressor gene, while the Ki-67 antigen is present on proliferating cells. The test tissue slice is a fixed, paraffin-embedded slice obtained from a mucous membrane close to a squamous cell carcinoma of the oral mucosa. Antigen retrieval is performed by immersing the tissue slice in 0.01M citric acid buffer with pH 6.0 and transferring the system to an autoclave to be heated. For the detection of bound primary antibodies, a polymer reagent or ChemMate Envision is used. Coupling pigments used in combination with HRP are SG for the detection of p53 protein, and AEC for the detection of Ki-67 antigen.

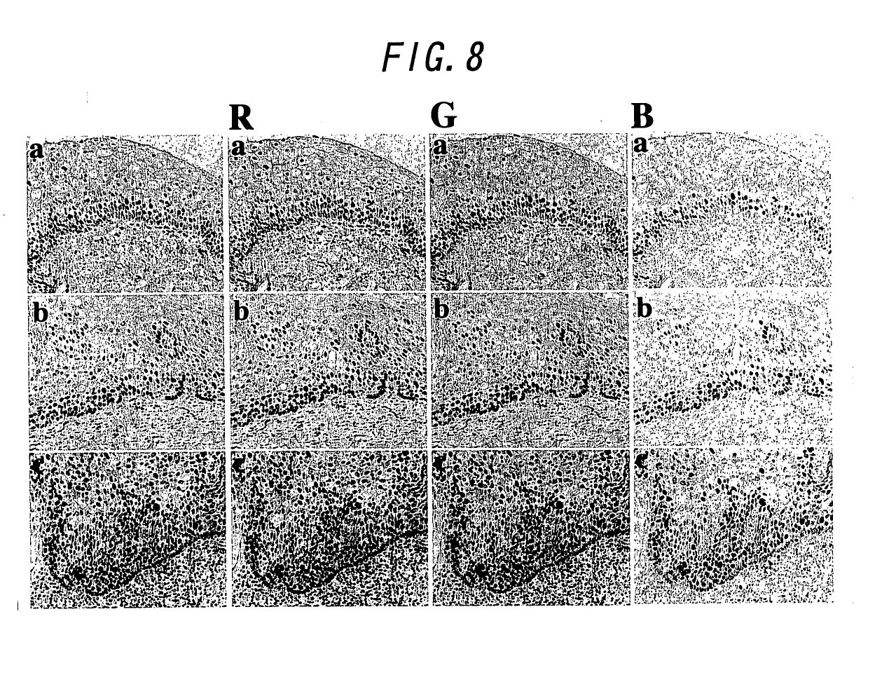

FIG. 8 shows the photographic images of the stained tissues. In FIG. 8, a first row of photographs a represent the images of a hyperplastic lesion of the squamous epithelium; a second row of photographs b those of a dysplastic lesion of the squamous epithelium; and a third row of photographs c those of an extremely displastic or carcinomatous lesion of the squamous epithelium. The excess accumulation of p53 protein in cell nuclei is stained bluish black with SG, and those nuclei stain positively in the G component image. The nuclei of the proliferating cells on which the Ki-67 antigen is present is stained red with AEC, and stains positively in the B component image. The inventive immunohistochemical staining gives the distinctive images of p53 protein and Ki67 antigen which characterize the process of the neoplastic formation of the squamous epithelium from its hyperplastic stage to extremely hyperplastic or carcinomatous stage.

[Procedures and Reagents Performed and Used in Example 7]

10% Buffered Formalin

A 4 g of sodium phosphate monobasic dihydrates and 26 g of sodium phosphate dibasic 12 hydrates are dissolved in 900 ml of ion-exchanged water, to which 100 ml of special grade formaldehyde solution (formalin) is added.

Antigen Retrieval

A fixed tissue slice removed of paraffin is treated with an enzyme such as trypsin, or immersed in a buffer such as 0.0M citric acid buffer with pH 6.0, and heated. Heating may take place in a microwave heater, autoclave, pressure cooker, warm water bath, etc. The enzymatic and heating treatments may be changed according to the type of target antigens.

0.01M Citric Acid Buffer with pH 6.0

A 2.1 g of citric acid monohydrate and 2.94 g of trisodium citrate dihydrate are dissolved in 100 ml of ion-exchanged water while being heated in an autoclave, and to the resulting solution, 10N sodium hydroxide is added to adjust its pH to pH 6.0, to which deionized water is added to 100 ml. This served as a 10-fold concentrated stock solution. Prior to use, an aliquot extracted from the stock solution is diluted 10 fold with ion-exchanged water.

Anti p53 Protein Antibody

The used antibody is a mouse anti human p53 protein antibody (NCL-p53-1801) available from Novocastra. The product provided by Novocastra is diluted 100 fold by the addition of DakoChemMate antibody diluent (S2022) prior to use.

Anti Ki-67 Antibody

The used antibody is a mouse anti human Ki-67 antibody (MIB-1, M7240) available from DakoCytomation. The product provided by DakoCytomation is diluted 50 fold by the addition of DakoChemMate antibody diluent (S2022) prior to use.

Polymer Reagent

This is a reagent consisting of a dextran polymer to which a secondary antibody and HRP are directly linked, and is provided by DakoCytomation under the trade name of a ChemMate Envision reagent.

Aminoethylcarbazole (AEC)

3-Aminoethyl-9-ethylcarbazole (Sigma) is dissolved in dimethyl-sulfoxide (DMSO) to give a 0.4% solution. This solution serves as a stock solution. A 5 ml of an aliquot is mixed with 95 ml of 0.1M acetic acid buffer with pH 5.0 to which 20 μl of pure hydrogen peroxide is added, and the resulting solution is filtered to be used. The 0.1M acetic acid buffer with pH 5.0 is obtained as follows. A 11.5 ml of 99% acetic acid is dissolved in ion-exchanged water to 1 L. A 14.8 ml of the resulting solution, and 16.4 g of sodium acetate or 27.2 g of sodium acetate trihydrate are dissolved in ion-exchanged water to 1 L, to which 35.2 ml of 0.2M sodium acetate is added, and the resulting solution is diluted with ion-exchanged water to 100 ml to give the desired acetic acid buffer. The same buffer is available in a kit provided by DakoCytomation.

SG

This coupling pigment developing a color through the reaction with HRP is available in a kit from Vector under the trade name of SK-4700.

The present invention is advantageous because of its providing a method for detecting antigens which greatly reduces the time necessary for the detection, and allows the selection of appropriate pigments used for the staining of antigens, and is suitable for the automated staining of antigens.

The present invention will greatly contribute to the spread of multiplicated immunohistochemical staining, and pave the way for the practical application of multiplicated immunohistochemistry in the diagnosis of cancer development because the inventive method can trace the development of a cancer by following two or more labeled antigens in that cancer.

1. A method for detecting two or more antigens based on multiplicated immunohistochemical staining using colored products or fluorescent dyes for visualizing the multiple antigens wherein:
an inhibitory step against non-specific reactions is performed for coating, with a non-specific reaction inhibiting substance, non-specific reactive substances which would otherwise react with primary antibodies in a non-specific manner, thereby inhibiting the non-specific reaction of those non-specific reactive substances with primary antibodies.

2. A method according to claim 1 wherein the inhibitory step against non-specific reactions is performed before a primary antibody is allowed to react with a first antigen.

3. A method according to claim 1 wherein the inhibitory step against non-specific reactions comprises treating a test sample with at least one chosen from the group comprising serum derived from an animal species from which a secondary antibody is prepared, skimmed milk, non-fat milk and casein solution.

4. A method according to claim 1 wherein the casein treatment uses a solution containing casein at 0.025 to 2.5%.

5. A method according to claim 1 wherein immuno-reactive substances bound to antibodies other than immuno-reactive products linked to colored products or fluorescent dyes used for the detection of antigens other than the lastly detected antigen, are removed by means of a buffer with a pH adjusted to pH 3 or lower, or pH 9 or higher.

6. A method according to claim 5 further comprising a step for cleaning a test sample using heated cleaning solution so that the test sample is removed of non-specific reaction products and other residual reaction products.

7. A method according to claim 6 wherein the temperature of the heated cleaning solution is in the range of 25 to 60° C.

8. A method according to claim 5 wherein removal of immuno-reactive substances other than immuno-reactive products linked to colored products or fluorescent dyes, is performed each time after the detection of an antigen till the detection of the last antigen.

9. A method according to claim 1 wherein the immunohistochemical staining uses plural pigments having different component profiles, and plural antigens are discriminated based on the different component profiles of the pigments.

10. A method according to claim 9 wherein the different component profile of plural pigments is expressed as a combination of R (red), B (blue) and G (green) components.

11. A method according to claim 1 wherein the colored product or fluorescent dye is a substance allowing visualization.

12. A method according to claim 11 wherein the substance allowing visualization comprises a coupling substance which becomes colored or emits light as a result of an immunohistochemical reaction where a labeling enzyme reacts with a substrate.

13. A method according to claim 11 wherein the substance allowing visualization is at least one chosen from the group comprising horseradish peroxidase (HRP), alkaline phosphatase (ALP), glucose oxidase, and beta-galactosidase.

14. A method according to claim 1 comprising designing a program for the execution of test procedures such as the selection of reaction solutions, reaction durations and washing times, and installing the program in an automated immunohistochemical staining system for the automated execution of immunohistochemical staining.
